(19) United States
(12) Patent Application Publication
(10) Pub. No.: US 2002/0098109 A1
(43) Pub. Date: Jul. 25, 2002

(54) Method and apparatus for producing purified or ozone enriched air to remove contaminants from fluids

(76) Inventors: Jerry Nelson, Warwick, RI (US); Craig Andrews, Cambridge, MA (US)

Correspondence Address: EPSTEIN, EDELL, SHAPIRO, FINNAN & LYTLE, LLC, Suite 400, 1901 Research Boulevard, Rockville, MD, 20850-3164, US

(21) Appl. No.: 09/912,546

(22) Filed: Jul. 26, 2001

Related U.S. Application Data
(63) Continuation of application No. 09/186,990, filed on Nov. 5, 1998, now abandoned.
(63) Continuation of application No. 09/156,422, filed on Sep. 18, 1998, now abandoned.
Application No. 09/156,422 is a continuation-in-part of application No. 08/932,101, filed on Sep. 17, 1997, now abandoned.
(60) Provisional application No. 60/064,348, filed on Nov. 5, 1997.
(60) Provisional application No. 60/064,520, filed on Nov. 5, 1997.
(60) Provisional application No. 60/094,574, filed on Jul. 29, 1998.

(51) Int. Cl. A61L 9/20; A61L 9/015; A61L 2/18; C02F 1/32; C02F 1/50

(52) U.S. Cl. 422/5; 422/24; 422/28; 422/121; 422/123; 422/124; 210/198.1; 210/748; 210/760; 96/224; 96/226

(57) ABSTRACT

A method and apparatus for producing purified or ozone enriched air to remove contaminants from fluids is accomplished by exposing an air stream from a surrounding area to ozone generating ultra-violet (UV) radiation to generate ozone in a system ozone chamber. The ozone chamber is configured to reduce through-flow velocity and provide time for the ozone to mix with the air and oxidize contaminants. The air stream subsequently enters a germicidal chamber and is exposed to germicidal UV radiation to destroy bacteria and ozone in the air stream resulting in sterilized air. The radiation source may include an end-cap that interfaces guiding mechanisms to align the end-cap for power connections, and/or controls emission of ozone generating radiation to control production of ozone. Further, the system may include an additional germicidal chamber that exposes an air stream to germicidal radiation prior to treatment within the ozone chamber. Moreover, the system may be configured to include a baffling arrangement to control air through-flow velocity, or may be implemented by a cartridge arrangement, whereby a cartridge housing the chambers and radiation sources is periodically replaced. The system may further be configured for installation within a wall or ceiling, or may be utilized to remove contaminants from and/or ozonate liquids. In addition, the air sterilization systems may be utilized within air treatment systems to remove contaminants from an air stream within these systems.

### CROSS-REFERENCE TO RELATED APPLICATIONS

This application is a continuation-in-part of copending U.S. patent application Ser. No. 09/156,422, entitled “Method and Apparatus for Producing Purified or Ozone Enriched Air”, filed Sep. 18, 1998, which is a continuation-in-part of U.S. patent application Ser. No. 08/932,101, entitled “Method and Apparatus for Removing Contaminants from a Contaminated Air Stream”, filed on Sep. 17, 1997. In addition, this application claims priority from U.S. Provisional Patent Application Serial No. 60/064,348, entitled “Method and Apparatus for Producing Purified or Ozone Enriched Air to Remove Contaminants from Fluids”, filed on Nov. 5, 1997, from U.S. Provisional Patent Application Serial No. 60/064,520, entitled “Method and Apparatus for Removing Contaminants from Air Streams Within Air Treatment Systems,” filed on Nov. 5, 1997, and from U.S. Provisional Patent Application Serial No. 60/094,574, entitled “Method and Apparatus for Producing Purified or Ozone Enriched Air to Remove Contaminants from Objects”, filed on Jul. 29, 1998. The disclosures in the above-referenced patent applications are incorporated herein by reference in their entireties.

### BACKGROUND OF THE INVENTION

1. Technical Field

The present invention pertains to a method and apparatus for producing purified or ozone enriched air to remove contaminants from fluids. In particular, the present invention pertains to a method and apparatus for exposing a contaminated fluid stream to ozone and germicidal radiation to remove contaminants from that fluid stream to produce purified fluid. In addition, the present invention may be disposed within an air treatment system (e.g., HVAC system, humidifier, heating and/or air conditioning units, etc.) to remove contaminants from air streams within those air treatment systems and return purified air to a surrounding environment.

2. Discussion of Related Art

Currently, there are numerous devices known as deodorizing machines utilizing ozone and/or ultraviolet (UV) radiation to sanitize and deodorize air in a treated space (i.e., typically a room). Generally, these devices generate large amounts of ozone gas to attain the ozone concentration level necessary to facilitate deodorizing and sterilizing the air. Since ozone concentration levels required for sterilization are sufficiently high to be dangerous to people and/or animals, the use of these devices is typically limited to odors whose removal is difficult (e.g., smoke from fires, organic material spilled on clothing, etc.). Further, when the devices are used in the proximity of people and/or animals, health authorities require that ozone concentrations be reduced to safe levels. However, these reduced or “safe” levels tend to be too low to effectively deodorize and clean the air. Moreover, such devices typically use the germicidal qualities of the ultraviolet radiation to destroy bacteria in the air, but generally either expose the treated space to high levels of radiation, thereby posing health risks to people and/or animals, such as eye trauma and skin lesions, or use very low levels of radiation requiring long exposure times.

The prior art attempts to obviate the aforementioned problems by exposing air from the treated space to ozone or UV radiation internally of a device to thereby shield against the above-mentioned harmful effects. For example, Burt (U.S. Pat. No. 3,486,308) discloses an air treatment device having a UV radiation source to sterilize air and a plurality of baffles disposed within the interior of the device housing. The baffles increase an air flow path within the device beyond the dimensions of the device housing to expose the air to radiation for greater periods of time. The UV source produces radiation at a particular intensity to avoid production of ozone.

Japanese Publication JP 1-224030 discloses an air cleaner including an ozone generating section, on ozone-air mixing section and a filter section. The filter section may include a pair of filters having an alkaline component and ozone-purifying material, respectively. Alternatively, the filter section may include a single filter having both an alkaline component and ozone-purifying material to clean air. The air cleaner further includes a winding air flow path for the air stream to traverse during cleaning.

The prior art devices disclosed in the Burt patent and Japanese Publication suffer from several disadvantages. In particular, the Burt device does not utilize ozone, thereby typically only removing bacterial contaminants (e.g., germs) within an air stream and enabling non-bacterial or other contaminants, such as odor causing contaminants, to be returned to a surrounding environment. Conversely, the air cleaner disclosed in the Japanese Publication employs only ozone to clean the air, thereby possibly destroying only a portion of bacterial contaminants within the air stream while returning residual bacterial contaminants to a surrounding environment.

The prior art attempted to overcome the above mentioned disadvantages by employing ozone in combination with UV radiation to remove virtually all contaminants from an air stream. In particular, Chesney (U.S. Pat. No. 2,150,263) discloses a system for internally cleaning, sterilizing and conditioning air within the system. A stream of air is washed and subsequently exposed to UV radiation which generates ozone such that the combination of UV radiation and ozone destroys virtually all bacteria in the air stream. Excess ozone is removed via pumps and utilized for various purposes.

Hirai (U.S. Pat. No. 5,015,442) discloses an air sterilizing and deodorizing system wherein UV radiation generates ozone to oxidize and decompose odor-causing components in the air. The ozone is then removed by a catalyzer in conjunction with, and prior to, germicidal UV radiation where the UV radiation also removes germs and sterilizes the air.

Monagan (U.S. Pat. No. 5,601,786) discloses an air purifier including a housing having an irradiation chamber, an air inlet for directing air into the irradiation chamber, a radiation source disposed within the irradiation chamber and an air outlet formed in the housing for discharging air to the environment. The radiation source preferably emits ozone-producing radiation within one wavelength interval, and germicidal radiation within another wavelength interval, whereby the emitted radiation serves to destroy microorganisms and deodorize the air.

LeVay et al (U.S. Pat. No. 5,614,151) discloses an electrodless sterilizer using ultraviolet and/or ozone. The sterilizer includes an energy source to excite a gas contained within a bulb and produce ultraviolet radiation, preferably strongest at 253.7 nanometers, that may be utilized to sanitize substances. Further, the radiation may be used to generate ozone that, either alone or in combination with the radiation, may sanitize substances. The bulb may be shaped to enable substances (e.g., liquid) to pass through the bulb for sterilization, or to enclose and shield objects (e.g., small articles) within the bulb from the energy source. Moreover, the bulb may be located at the end of a waveguide, or radiation may be transmitted from the bulb via an optic feed to sanitize inaccessible surfaces of substances. In addition, an ozone generator may be utilized to apply ozone to an external substance, whereby flexible hosing connected to the ozone generator includes a nozzle to control discharge of ozone onto a substance.

The Chesney, Hirai, Monagan and LeVay et al systems suffer from several disadvantages. Specifically, the Chesney and LeVay et al systems typically utilize a single wavelength of UV radiation (e.g., approximately 254 nanometers) which may not be optimal for both generating ozone and destroying bacteria. In fact, this wavelength is generally utilized for its germicidal effects and tends to destroy ozone, thereby degrading the effect of ozone within the air stream. Although the Monagan system utilizes a radiation source emitting ozone-producing and germicidal radiation, an air stream is exposed to each type of radiation simultaneously, thereby enabling the germicidal radiation to destroy produced ozone and degrade the effect of ozone within the air stream. Further, the Chesney system includes a relatively lengthy compartment for treating air, thereby increasing the size and cost of the system. The Hirai system typically utilizes independent radiation sources to generate ozone and germicidal radiation, thereby increasing system cost and complexity. Moreover, the Hirai system does not provide a safety feature where the ozone generating source may be operable when the germicidal or ozone removing source becomes inoperable, thereby leading to emissions of dangerous ozone concentrations from the system. In addition, the Hirai system employs a relatively short, narrow area for ozone generation, while the Monagan system includes a radiation source having adjacent portions emitting ozone generating and germicidal radiation, and a substantially linear path disposed within an irradiation chamber for an air stream to traverse the radiation source. Thus, the effects of ozone within an air stream in the Hirai and Monagan systems are degraded since there is generally a minimal amount of time and/or space for the ozone to interact with the air prior to exposure to germicidal radiation.

Although the LeVay et al system may sanitize substances via ozone and ultraviolet radiation, the ozone is typically generated by a single wavelength of radiation (e.g., approximately 254 nanometers) that tends to destroy ozone as described above, thereby minimizing the effects of ozone on the substance. Further, the LeVay et al system sanitizes a liquid substance by introducing ozone into the liquid subsequent to exposure of that liquid to germicidal radiation, thereby enabling the liquid to contain ozone concentration levels sufficient to cause possible harm to people and/or animals that contact the treated liquid. The LeVay et al patent further discloses systems for applying ultraviolet radiation or ozone to surfaces of substances external of those systems. The radiation may be applied to the external substance via a light pipe or optic feed, while ozone may be applied via a nozzle disposed at an end of flexible hosing attached to an ozone generator. However, these devices may not fully expose the substance surfaces to the ultraviolet radiation or ozone, thereby incompletely sanitizing the substance. Moreover, the ultraviolet radiation or ozone is applied to the substance surfaces typically without preventive or containment measures, thereby enabling radiation and ozone to be released to the surrounding environment and cause possible harm to people and/or animals in the vicinity of the substance as described above.

### OBJECTS AND SUMMARY OF THE INVENTION

Accordingly, it is an object of the present invention to expose fluids to ozone and ultraviolet radiation to remove contaminants from the fluids.

It is another object of the present invention to reduce costs and minimize the size of an ozone generating chamber within a system for removing contaminants from fluids by utilizing an ozone chamber configured to reduce air through-flow velocity (i.e., increase the amount of time air resides within the ozone chamber to reduce air flow velocity through the ozone chamber) to enable ozone generated in the ozone chamber to interact and mix with an air stream to produce ozone enriched air to remove contaminants from fluids.

Yet another object of the present invention is to maintain ozone concentration levels at low or “safe” levels in a system for removing contaminants from fluids by utilizing a single radiation source in the system to emit radiation of different wavelengths from different sections of the source to generate ozone and perform germicidal functions on the fluid, respectively. The entire single radiation source can become disabled only as a unit, thereby preventing generation of ozone when the germicidal radiation or ozone-removing section is inoperable.

Still another object of the present invention is to control ozone concentration levels by utilizing a radiation source end-cap having various configurations to regulate emission of ozone generating radiation from the radiation source.

A further object of the present invention is to utilize replaceable cartridges with a system for removing contaminants from fluids to facilitate versatility and easy maintenance of the system.

Yet another object of the present invention is to remove contaminants from air streams within air treatment systems (e.g., HVAC system, humidifier, heating and/or air conditioning units, etc.) and return purified air to a surrounding environment.

The aforesaid objects are achieved individually and in combination, and it is not intended that the present invention be construed as requiring two or more of the objects to be combined unless expressly required by the claims attached hereto.

According to the present invention, a method and apparatus for removing contaminants from a contaminated air stream is accomplished by a system in which air is drawn in as a stream into the system housing toward its base and flows through an ozone generating chamber. An ozone generating ultraviolet (UV) radiation source disposed within the ozone chamber emits ultraviolet radiation having a wavelength of approximately 185 nanometers to irradiate the air and generate ozone which oxidizes contaminants (e.g., bacteria, virus, odor-causing element, etc.) residing in the air stream. The ozone chamber is typically configured to include winding or other types of air flow paths, or to induce a vortical air flow, to reduce air through-flow velocity and maintain the air stream within the ozone chamber for a residence time sufficient for the ozone to interact with the air. Subsequent to traversing the ozone chamber, the air stream enters a germicidal chamber disposed adjacent the ozone chamber. The germicidal chamber may also be configured to have winding or other types of air flow paths, and includes a germicidal UV radiation source. The germicidal UV radiation source irradiates the air stream and destroys bacteria and breaks down ozone residing therein. The germicidal UV radiation source generates radiation having a wavelength of approximately 254 nanometers to destroy bacteria, viruses, mold spores and ozone remaining after the interaction of air and ozone in the ozone chamber. The radiation source typically includes a single combination UV radiation emitting bulb with different sections of the bulb emitting radiation of different respective wavelengths (e.g., 185 and 254 nanometers). The different sections of the bulb are disposed in the corresponding ozone and germicidal chambers. Alternatively, the radiation sources may all be implemented by separate independent bulbs emitting radiation having wavelengths of approximately 185 or 254 nanometers depending upon the chamber in which the bulb is disposed. The bulbs may be powered by a conventional AC ballast (for use in stationary areas), or a conventional DC ballast connected to a battery or other DC power source to enable the system to be portable and used in mobile environments (e.g., cars, boats, trucks, trailers, etc.). In addition, the combination bulb may further include end-caps of various configurations to align the bulb for power connections and/or to regulate emission of ozone generating radiation and control production of ozone.

The resulting sterilized air from the germicidal chamber may pass through a catalytic converter disposed adjacent the germicidal chamber to remove any remaining ozone by either converting the ozone back to oxygen, or filtering the ozone from the air stream. An internal fan disposed adjacent the ozone chamber draws air into the system from the base and through the chambers. The system is typically constructed of injection molded plastic, whereby the system housing includes two symmetrical halves. Alternatively, the system may be constructed of foam having a plastic or other suitable rigid covering. Symmetrical portions of the ozone and germicidal chamber configurations are molded into the respective symmetrical halves such that the symmetrical halves are connected (e.g., snapped or otherwise fastened together) to form the system. In addition, the system may include a bulb holder that is disposed on the system top surface and extends into the system interior to secure the bulb. The bulb holder extracts the bulb from the system upon removing the bulb holder from the system top surface.

Moreover, the system may include an additional germicidal chamber. Specifically, the system has substantially the same configuration described above except that that the system ozone chamber is disposed between a pair of germicidal chambers. The initial germicidal chamber exposes an air stream to germicidal radiation to remove contaminants from that stream, while the subsequent ozone and germicidal chambers treat the stream in substantially the same manner described above. A combination bulb emitting germicidal radiation from two different bulb sections and ozone generating radiation from an additional bulb section is disposed with the bulb sections positioned within the corresponding germicidal and ozone chambers. A fan, disposed proximate the initial germicidal chamber, draws air through the system.

The system may further be configured to utilize a baffling arrangement to control air through-flow velocity through the system. In particular, the system is substantially similar to, and functions in substantially the same manner as, the two chamber system described above, except that the system includes a series of baffles to form a serpentine path through the system. The baffles include an alternating pattern of openings that collectively direct an air stream in a winding pattern through the system chambers to remove contaminants from that stream.

The system may alternatively be configured to utilize a replaceable cartridge. Specifically, a stationary base is mounted in a desired area, whereby a replaceable cartridge is attached to the base. The base contains the system electrical components (e.g., fan, ballast, etc.), while the cartridge houses the chambers and radiation source. The cartridge may further be disposed in a plenum without the base or a fan, whereby the cartridge is connected to a power source and plenum air flow directs air through the system. The cartridge may be of various shapes and sizes and is periodically replaced, thereby facilitating versatility and easy maintenance of the system.

The system may be configured for installation within a wall or ceiling. Specifically, a ceiling or wall unit has a similar configuration as described above and includes a pair of ozone chambers and a pair of germicidal chambers. The ozone and germicidal chambers within each pair are respectively disposed adjacent each other, and function in parallel in substantially the same manner described above. The ozone and germicidal chambers are each constructed within a block of foam wherein the ozone chambers each include a winding path to reduce air through-flow velocity and enable generated ozone to mix and interact with an air stream. Air is directed by the ozone chambers to corresponding germicidal chambers to remove bacteria from the air stream as described above. The germicidal chambers are disposed adjacent a corresponding ozone chamber and share a common area formed within the foam block. A combination bulb and an additional radiation source emitting germicidal radiation are disposed within each germicidal chamber, while a fan, disposed proximate the germicidal chambers, draws air through the system. Alternatively, the ceiling or wall unit may include a single ozone chamber and a single germicidal chamber formed in the foam block, and a plurality of combination bulbs to treat the air in substantially the same manner described above.

The system may be further utilized to remove contaminants from liquids by exposing the liquid to ozone and germicidal radiation. Specifically, a system ozone chamber produces ozone and includes a tortuous or winding path to enable the produced ozone to interact with the air. The ozonated air is injected into the liquid, while a system germicidal chamber exposes the ozonated liquid to germicidal radiation to remove residual contaminants and ozone. A combination radiation source is typically utilized to provide ozone generating and germicidal radiation within the chambers. The system may be disposed along pipelines or to a faucet to purify drinking or other water within a dwelling or other building. Moreover, the system may ozonate water for application to food or other items to remove contaminants from those items.

In addition, the air sterilization systems described above may be utilized within air treatment systems (e.g., HVAC system, humidifier, heating and/or air conditioning units, etc.) to remove contaminants from an air stream within these air treatment systems and return purified air to the surrounding environment.

The above and still further objects, features and advantages of the present invention will become apparent upon consideration of the following detailed description of specific embodiments thereof, particularly when taken in conjunction with the accompanying drawings wherein like reference numerals in the various figures are utilized to designate like components.

### BRIEF DESCRIPTION OF THE DRAWINGS

FIG. 1 is a side view in elevation of a system for removing contaminants from a contaminated air stream to produce purified or ozone enriched air including a combination exhaust vent and bulb holder to facilitate placement and removal of an ultra-violet (UV) radiation emitting bulb within the system interior according to the present invention.

FIG. 2 is a top view in plan of the combination exhaust vent and bulb holder of the system of FIG. 1.

FIG. 3 is a side view in elevation and partial section of the system of FIG. 1.

FIG. 4 is an exploded view in perspective of an end-cap and associated connector for the ultra-violet (UV) radiation emitting bulb of the system of FIG. 1 according to the present invention.

FIG. 5 is an exploded view in perspective of an alternative embodiment of the end-cap and associated connector of FIG. 4.

FIG. 6 is a view in perspective of an end-cap for the ultra-violet (UV) radiation emitting bulb of the system of FIG. 1 that controls intensity of radiation emitted from the bulb to regulate production of ozone according to the present invention.

FIG. 7 is a view in perspective of an alternative embodiment of the end-cap of FIG. 6.

FIG. 8 is a view in perspective of a combination ultra-violet (UV) radiation emitting bulb including an end-cap having windows to regulate emission of ozone generating radiation according to the present invention.

FIG. 9 is a side view in elevation and partial section of a portion of an alternative embodiment of the system of FIG. 1 including an additional germicidal chamber to remove contaminants from a contaminated air stream to produce purified or ozone enriched air.

FIG. 10 is a view in perspective of an internal structure of a system for producing purified or ozone enriched air including a series of baffles forming a tortuous or serpentine air flow path through the system according to the present invention.

FIG. 11 is an exploded view in perspective of an alternative embodiment of the system of FIG. 10.

FIG. 12 is an exploded view in perspective of a system including a base and a replaceable cartridge having ozone and germicidal chambers and a radiation source for producing purified or ozone enriched air according to the present invention.

FIG. 13 is a view in perspective of the rear portion of the cartridge of the system of FIG. 12.

FIG. 14 is a view in perspective of a cartridge component for forming the cartridge of the system of FIG. 12.

FIG. 15 is an exploded view in perspective and partial section of the cartridge of the system of FIG. 12 diagrammatically illustrating the air flow path through the cartridge.

FIG. 16 is a view in elevation and partial section of an alternative configuration for the cartridge of the system of FIG. 12.

FIG. 17 is a view in elevation and partial section of another configuration for the cartridge of the system of FIG. 12.

FIG. 18 is a view in perspective of the replaceable cartridge of the system of FIG. 12 configured for use within plenums of vehicles or other locations (e.g., ducts) according to the present invention.

FIG. 19 is a view in perspective of the rear portion of the cartridge of the system of FIG. 18.

FIG. 20 is a view in perspective of an end-cap for use with the cartridge radiation source of the system of FIG. 12 according to the present invention.

FIG. 21 is a view in perspective and partial section of the end-cap of FIG. 20.

FIG. 22 is a view in elevation and partial section of the end-cap of FIG. 20 disposed within the cartridge of the system of FIG. 12 to interface the cartridge radiation source according to the present invention.

FIG. 23 is an exploded view in perspective of a system for removing contaminants from a contaminated air stream to produce purified or ozone enriched air, typically configured for installation within a ceiling or wall according to the present invention.

FIG. 24 is a view in perspective of an end-cap for an ultra-violet (UV) radiation emitting bulb of the system of FIG. 23.

FIG. 25 is a top view in plan of a portion of another embodiment of the system of FIG. 23 including a single ozone chamber and a single germicidal chamber to remove contaminants form a contaminated air stream to produce purified or ozone enriched air.

FIG. 26 is a view in elevation and partial section of a system for removing contaminants from liquid flowing within a pipeline according to the present invention.

FIG. 27 is a side view in elevation and partial section of a sink utilizing a system to remove contaminants from tap water as the tap water flows to or from the sink faucet according to the present invention.

FIG. 28 is a side view in elevation and partial section of a sink utilizing the system of FIG. 27 for ozonating water to apply ozonated water to food or other items to remove contaminants from those items.

FIG. 29 is a view in elevation and partial section of a portion of an air treatment system including a humidifier employing a drum to introduce moisture into an air stream, and an air sterilization system to remove contaminants from the air stream and enable the air treatment system to return purified treated air to a surrounding environment according to the present invention.

FIG. 30 is a view in elevation and partial section of a portion of an air treatment system including a humidifier employing a spray nozzle to introduce moisture into an air stream, and an air sterilization system to enable the air treatment system to return purified treated air to a surrounding environment according to the present invention.

FIG. 31 is a side view in elevation and partial section of an exemplary stand alone humidifier including an air sterilization system for removing contaminants from an air stream to enable the humidifier to return purified treated air to a surrounding environment according to the present invention.

### DETAILED DESCRIPTION OF THE PREFERRED EMBODIMENTS

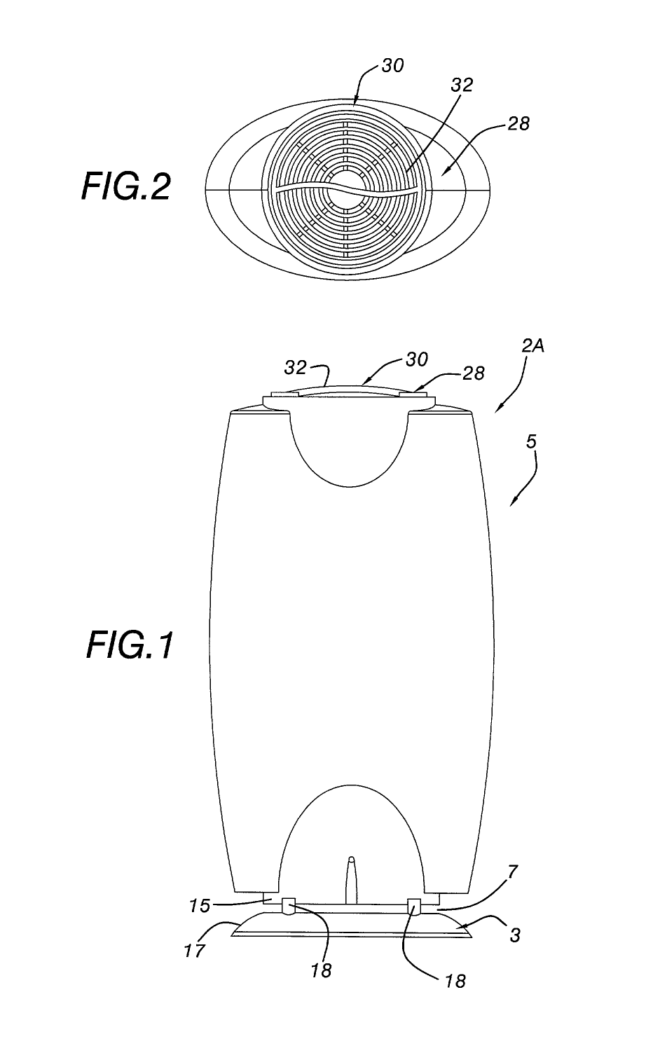

A system 2a for removing contaminants from a contaminated air stream to produce purified or ozone enriched air including a combination exhaust vent and bulb holder is illustrated in FIGS. 1-3. Specifically, system 2a includes a generally cylindrical housing 5 extending from a base 3, ozone and germicidal chambers 8, 16, a UV radiation source 36, typically implemented by a combination ultraviolet radiation emitting bulb and typically disposed at the approximate center of the ozone and germicidal chambers, a ballast (not shown), preferably conventional, for supplying current to radiation source 36, and an internal fan (not shown) for drawing air through the system. The radiation source may be implemented by a single bulb having an ozone section 12 and germicidal section 14 emitting radiation at different wavelengths (e.g., approximately 185 and 254 nanometers) from the ozone and germicidal sections, respectively. The bulb typically includes coated or specialized glass or other material that filters radiation to enable specific sections of the bulb to emit radiation having particular wavelengths (e.g., ozone section 12 and germicidal section 14). Alternatively, the radiation source may be implemented by two independent bulbs disposed in the respective ozone and germicidal chambers, whereby each independent bulb emits radiation having a particular wavelength (e.g., approximately 185 or 254 nanometers). Housing 5 includes a middle portion that has cross-sectional dimensions slightly larger than the cross-sectional dimensions of the housing end portions such that the housing has a shape similar to a barrel. Base 3 is typically constructed of upper and lower supports 15, 17 (FIG. 1), whereby the supports are attached to each other via legs or connectors 18 disposed between the supports. Lower support 17 serves as a stand for the system, while upper support 15 typically contains the system electrical components, such as a ballast and fan (not shown) for supplying current to the radiation source and directing air through the system, respectively. However, the fan may be disposed anywhere in the system capable of directing air through the system, while the electrical components may be disposed in the system in any a fashion. Legs 18 separate upper and lower supports 15, 17 by a slight distance to form an air intake 7 that serves to permit air to enter the system. Base 3 may alternatively be constructed of a single support configured to enable air to enter the system.

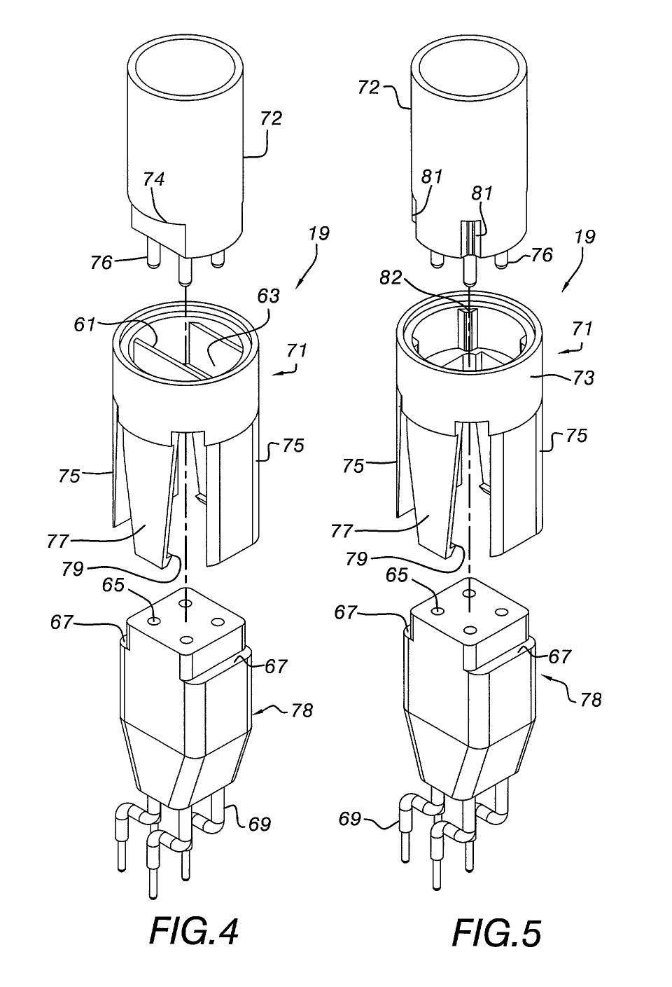

Air from a surrounding environment is drawn into the system through air intake 7 by the internal fan (not shown) and is directed via the housing internal structure to flow into ozone chamber 8, typically disposed above and adjacent the internal fan and air intake. Ozone chamber 8 includes ozone section 12 of radiation source 36 and a path 10 that serves to decrease air through-flow velocity (i.e., the path increases residence time of an air stream within the ozone chamber, thereby decreasing velocity of the air stream through the chamber) and enhance ozone distribution within the air stream. The end of radiation source 36 adjacent ozone section 12 is placed within a power connector 19 disposed at the approximate center of the bottom portion of the ozone chamber. The power connector may alternatively be disposed anywhere in the ozone chamber capable of receiving the end of the radiation source. It is to be understood that the terms “top”, “bottom”, “upper”, “lower”, “up”, “down”, “height”, “width”, “length”, “thickness”, “depth”, “front”, “rear”, “near”, “far”, “back”, “side”, “horizontal” and “vertical” are used herein merely to facilitate descriptions of points of reference and do not limit the present invention to any specific configuration or orientation. Power connector 19 provides current from a ballast (conventional and not shown) to radiation source 36, and may be implemented by any conventional or other type of connector, such as the connectors described below for FIGS. 4-5. The end of radiation source 36 adjacent germicidal section 14 is placed within a bulb holder 30 of an exhaust vent 28 whereby the exhaust vent is disposed on the system top surface with the bulb holder extending from the exhaust vent into the system interior. The radiation source extends from power connector 19 toward bulb holder 30 with the ozone and germicidal sections typically disposed at the approximate center of the respective ozone and germicidal chambers, however, the ozone and germicidal sections may be disposed in the respective ozone and germicidal chambers in any fashion. Alternatively, system 2a may be configured such that radiation source 36 has a portion of germicidal section 14 disposed within the ozone chamber to enable the path to combine the effects of ozone producing and germicidal radiation to further remove contaminants from the air stream and to control ozone concentration within the air stream (i.e., the greater the germicidal portion disposed in the ozone chamber, the lower the ozone concentration within the air stream).

Path 10 receives an air stream entering ozone chamber 8 from the approximate bottom center of the ozone chamber proximate ozone section 12 and transversely directs the air stream away from ozone section 12 toward housing 5. Ozone section 12 generates ozone within the air stream, while path 10 reduces air through-flow velocity and enables the ozone to mix and interact with the air stream to oxidize contaminants. A plurality of reversing passages 31 form path 10, whereby the passages are defined by spaces between a plurality of walls 20, 29. Walls 20, 29 are disposed within the ozone chamber between upper and lower ozone dividers 25, 27 that define the confines of the ozone chamber. Walls 20 each extend from an end of upper divider 25 substantially parallel to each other toward lower divider 27, whereby the length of each wall 20 is slightly less than the distance between the upper and lower dividers to form a gap that enables the air stream to enter and traverse succeeding passages 31. Similarly, walls 29 each extend from an intermediate portion of lower divider 27 such that ozone section 12 is disposed between walls 29 and walls 29 are disposed between walls 20. Walls 29 each extend from lower divider 27 toward upper divider 25, whereby the length of each wall 29 is slightly less than the distance between the upper and lower dividers to form a gap that enables the air stream to enter and traverse succeeding passages 31. The upper and lower ozone dividers maintain the air stream within ozone chamber 8, and isolate the ozone chamber from the remaining portions of the housing. Ozone dividers 25, 27 typically extend across the housing interior to prevent the air stream from bypassing portions of path 10. Lower divider 27 includes an opening toward its intermediate portion to permit the air stream to enter ozone chamber 8, while upper divider 25 is of sufficient size to form gaps between the upper divider periphery and housing 5 to permit air to enter germicidal chamber 16 from the ozone chamber. However, the air intake and upper and lower ozone dividers may be arranged in any manner to facilitate traversal of the ozone and germicidal chambers by an air stream. Housing 5 and its internal structural components may be constructed of injection molded plastic or other material and molded within substantially symmetrical halves of the housing. In other words, symmetrical portions of walls 20, 29, ozone dividers 25, 27 and the remaining structural components of housing 5 (e.g., the germicidal chamber) may be molded into corresponding halves of housing 5 such that when the halves are connected (e.g., the halves may be snapped together or connected utilizing any connection technique), the ozone chamber, path and other housing components are formed.

Upon entering ozone chamber 8 from air intake 7, the air stream traverses path 10 wherein the air through-flow velocity is reduced to enable ozone, generated by ozone section in 12, to mix with the air stream to oxidize and remove contaminants within the air stream. Further, when a portion of germicidal section 14 is disposed within the ozone chamber, radiation emitted from the germicidal section enhances removal of contaminants from the air stream. Once the air stream traverses path 10, the air stream leaves the ozone chamber and enters germicidal chamber 16. Germicidal chamber 16 includes germicidal section 14 of radiation source 36 that emits germicidal UV radiation to destroy contaminants and ozone within the air stream. Housing 5 may include reflective material within the germicidal chamber to enhance the germicidal effect of radiation emitted from germicidal section 14. The germicidal chamber typically shields a user from any visual UV light, and is isolated from the ozone chamber. The sterilized air from the germicidal chamber is exhausted from the system through exhaust vent 28 to the surrounding environment.

Exhaust vent 28 is substantially elliptical, but may be of any shape, and is disposed at the approximate center of the system top surface. Exhaust vent 28 includes bulb holder 30 having a user gripping portion 32 preferably disposed at the approximate center of the exhaust vent. Gripping portion 32 is typically substantially circular, but may be of any shape and may be disposed anywhere on the exhaust vent. Bulb holder 30 further includes a bulb receptacle 21 that typically extends from the approximate center of gripping portion 32 into the germicidal chamber to engage the end of radiation source 36 adjacent germicidal section 14 as described above. The receptacle may alternatively extend from any portion of gripping portion 32, and may include any type of clamp, brace, bracket, receptacle or other mechanism for engaging the radiation source. Bulb holder 30 facilitates removal and placement of radiation source 36 within the system interior. In particular, removal of radiation source 36 from the system interior is facilitated by extracting bulb holder 30 from the system via gripping portion 32. Since radiation source 36 is attached to the bulb holder, the radiation source is also extracted, thereby disconnecting the radiation source from power connector 19. Thus, the radiation source is disabled prior to removal from the system interior to prevent exposure to direct UV light. Conversely, placement of a TV bulb into the system is facilitated by disposing bulb holder 30, containing a UV bulb, back onto the system, via gripping portion 32, with the bulb extending into power connector 19. The bulb is enabled when the bulb is disposed within power connector 19 and gripping portion 32 is placed on the system top surface, thereby preventing exposure to direct UV light. System 2a may be of any shape or size with the chambers and path configured in any manner and the bulb holder disposed on the system in any fashion at any location. Further, the system may be mounted on a wall or other structure (e.g., typically including the fan and electrical components disposed within the system with or without the base), and may be utilized with an exhaust vent without the bulb holder. The housing and its internal structure may be constructed of any suitable material and, by way of example only, the system may include a height of approximately thirteen inches with the housing being constructed of injection molded plastic. The ozone generation and application of germicidal radiation may be controlled to produce ozone enriched air having a particular ozone concentration level for various applications as described below.

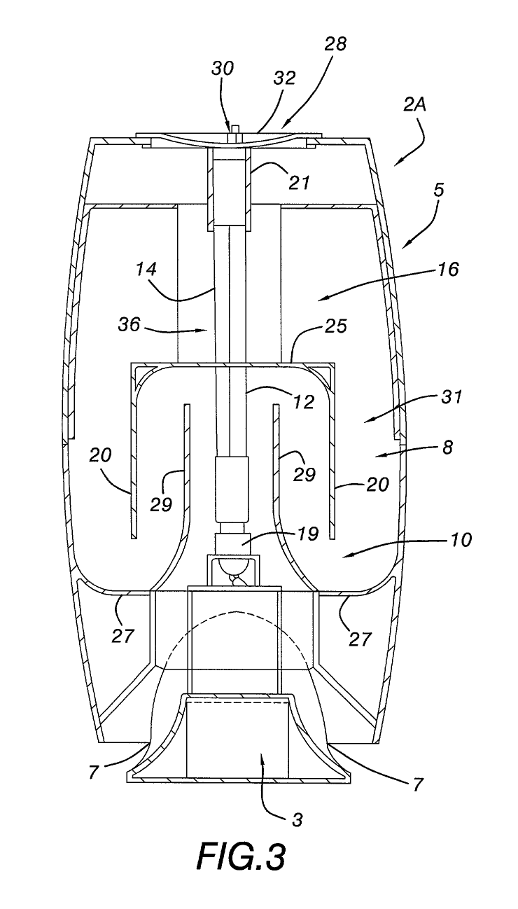

Power connector 19 of system 2a may be a custom connector to specifically interface radiation source 36 to a ballast as illustrated in FIG. 4. Specifically, an end-cap 72 is disposed at an end of radiation source 36 (FIG. 3) adjacent ozone section 12. The end-cap is shown generally cylindrical, but may be of any shape, and includes an open top portion for receiving the end of radiation source 36. The bottom portion of the end-cap is cut-off or truncated at opposing locations on the end-cap (e.g., angularly displaced by approximately 180°) to form a generally rectangular cross-section having rounded edges along the shorter rectangular cross-sectional dimension. The truncated cross-section extends from the bottom toward the top of the end-cap for approximately one-quarter of the end-cap height. An overhang 74 is formed proximate each of the locations on the end-cap where the truncated and non-truncated portions of the end-cap interface (i.e., the interface between the generally circular and rectangular cross-sections of the end-cap) since the non-truncated portion includes crosssectional dimensions greater than the cross-sectional dimensions of the truncated portion. A plurality of pins 76, preferably four, is disposed on and extends from the end-cap bottom. The pins are substantially cylindrical, but may be of any shape and any quantity (e.g., at least one), and accommodate wiring from radiation source 36 to interface a power plug 78 for connection to a ballast (not shown).

End-cap 72 is received within a female plug 71 that interfaces power plug 78. Female plug 71 includes a substantially cylindrical head 73 and a series of extensions 75, 77 alternately extending downward from the bottom periphery of head 73 to engage power plug 78. However, head 73 may be of any shape and has cross-sectional dimensions greater than end-cap 72 to receive the end-cap and enable the end-cap to interface power plug 78 as described below. Substantially rectangular dividers 61, 63 are disposed within and extend substantially in parallel across the interior confines of head 73. The dividers may alternatively be of any shape and are separated by a sufficient distance to receive the truncated portion of end-cap 72 between the dividers, while enabling overhangs 74 to engage the divider top surfaces in response to proper manipulation of the end-cap within head 73. In other words, dividers 61, 63 and overhangs 74 interact to form a guiding mechanism to enable alignment of end-cap 72 with power plug 78.

Extensions 75 of female plug 71 are substantially rectangular and taper in thickness toward their distal ends, while extensions 77 of female plug 71 are substantially trapezoidal and taper in width toward their distal ends. The distal portion of each extension 77 has a thickness slightly greater than the thickness of the remaining portions of that extension. The distal portion thickness of each extension 77 tapers distally toward the distal end. of that extension whereby a ledge or hook 79 is formed proximate the interface between a proximal portion and the thicker distal portion of the extension to engage power plug 78. However, extensions 75, 77 may be of any shape and may include any mechanism to engage the power plug. When radiation source 36 is disposed within system 2a as described above, the radiation source, and hence, end-cap 72, is manipulated such that the truncated portion of the end-cap resides between dividers 61, 63, while overhangs 74 engage dividers 61, 63 of head 73 to align pins 76 for connection to power plug 78.

Power plug 78 is a generally rectangular block having a top surface including receptacles 65, preferably four, for receiving corresponding pins 76 from end-cap 72, however, the power plug may be of any shape and may include any quantity of receptacles. Power plug 78 includes a substantially rectangular cross-section with an upper portion truncated or cut-off along the shorter rectangular cross-sectional dimension to form ledges 67. The proximal portion of power plug 78 tapers in width toward the power plug proximal end and interfaces wiring 69, typically including wiring for each pin 76, that respectively connects pins 76 to a ballast (not shown) to provide power to the radiation source. Power plug 78 is inserted within female plug 71 such that hooks 79 of extensions 77 engage the bottom portion of the power plug. The power plug is oriented within female plug 71 in a manner to receive pins 76 within receptacles 65 when end-cap 72 is properly oriented within head 73 as described above.

An alternative embodiment for power connector 19 of system 2a is illustrated in FIG. 5. Power connector 19 is substantially similar to the power connector described above for FIG. 4 except that a different guiding mechanism is implemented to align end-cap 72 with power plug 78. Specifically, end-cap 72 is generally cylindrical having an open top portion as described above. The bottom portion of the end-cap includes a series of substantially rectangular notches or recesses 81 extending from the end-cap bottom toward the end-cap top for approximately one-quarter of the end-cap height. The notches are angularly spaced from one another about the end-cap outer surface by approximately ninety degrees, and taper in width as the notches extend into the end-cap surface. Female plug 71 is substantially similar to the female plug described above and includes a substantially cylindrical head 73 having extensions 75, 77 alternately extending downward from the bottom periphery of head 73 to engage power plug 78 as described above. The distal portions of extensions 77 include hooks 79 to engage power plug 78 as described above. Head 73 includes a plurality of pegs or posts 82 of generally triangular cross-section disposed about the interior surface of head 73 and extending between the top and bottom portions of the head. Posts 82 are angularly spaced from one another about the head interior surface by approximately ninety degrees, and transversely extend from the head interior surface for a distance slightly less than the depth of notches 81. End-cap 72 is placed within female plug 71 and manipulated such that posts 82 engage notches 81. The notches and posts orient end-cap 72 within female plug 71 in a proper manner to align pins 76 for interfacing power plug 78 as described above. Alternatively, the notches and posts may be of any shape or size, may be of any quantity and may be disposed on the respective end-cap and female plug in any manner capable of aligning the end-cap with the power plug. Further, the end-cap and female plug may be configured with any structures in any manner capable of aligning the end-cap with the power plug.

Power plug 78 is substantially similar to the power plug described above and includes a series of receptacles 65 for receiving corresponding pins 76 of end-cap 72. Power plug 78 interfaces wiring 69 at its proximal end that respectively connects pins 76 to a ballast (not shown) to provide power to the radiation source as described above. Power plug 78 is inserted within female plug 71 such that hooks 79 of extensions 77 engage the bottom portion of the power plug as described above. Receptacles 65 receive corresponding pins 76 when end-cap 72 is properly oriented within head 73 via notches 81 and posts 82 as described above.

In order to utilize the guiding mechanisms of female plug 71 described above for radiation sources having conventional or other types of ends or connectors, an adapter may be utilized to interface these radiation sources to female plug 71 and power plug 78. For example, the adapter may be similar in configuration to the end-caps described above and interface terminals or wiring from a radiation source. The radiation source and adapter are manipulated as described above for proper connection to power plug 78 via female plug 71. The adapter may be similar in configuration to any of the end-cap embodiments described above (e.g., FIGS. 4 and 5), or may be any adapter capable of interfacing radiation source 36 to female plug 71 and power plug 78.

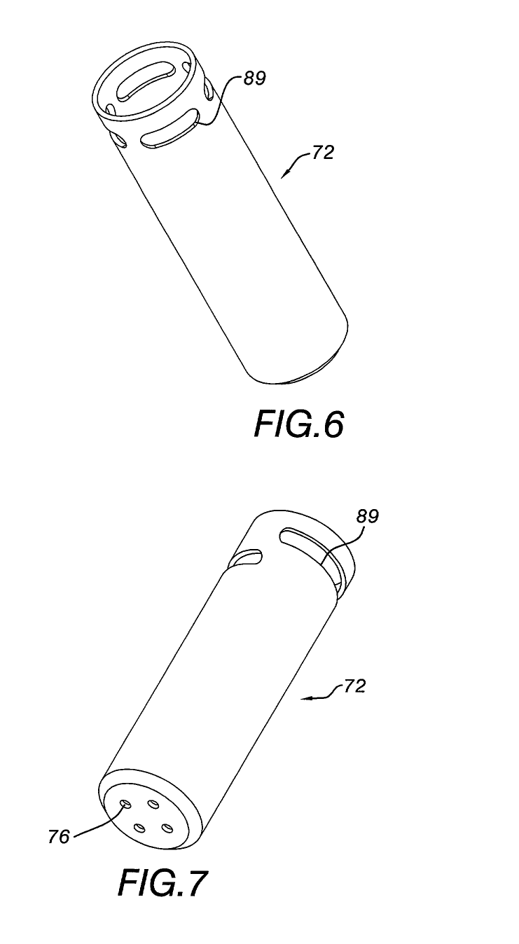

In addition, end-cap 72 may control production of ozone within ozone chamber 8 as illustrated in FIGS. 6-7. End-cap 72 is similar to the end-caps described above except that the end-cap is elongated to cover a portion of radiation source 36. The end-cap may be configured as described above for FIGS. 4-5 to implement the guiding mechanisms, but by way of example and to facilitate this description, the end-cap is illustrated in a configuration not employing those guiding mechanisms. Specifically, end-cap 72 is substantially cylindrical and elongated to extend along and cover ozone section 12 of radiation source 36, however, the end-cap may be of any shape or size. The end-cap includes an open top portion to receive ozone section 12 of radiation source 36 and is typically constructed of materials that block or prevent passage of radiation from the source. Slots 89 are defined in the end-cap to regulate the amount of radiation emitted in the ozone chamber (i.e., the amount of radiation permitted to pass from the bulb through the end-cap into the ozone chamber), thereby controlling ozone production. Slots 89 are typically elliptical and defined in end-cap 72 about the exterior end-cap surface in a non-overlapping manner angularly spaced a slight distance from each other toward the upper portion of the end-cap (FIG. 6). Alternatively, slots 89 may be defined about the end-cap exterior surface in an overlapping or helical fashion toward the upper portion of the end-cap (FIG. 7). However, the slots may be of any size or shape, may be of any quantity and may be defined in the end-cap in any fashion to facilitate particular radiation intensities within the ozone chamber to produce desired ozone concentrations. End-cap 72 may include predetermined slot arrangements to produce a desired ozone concentration level, or may include a particular slot arrangement that is used in conjunction with a radiation emitting bulb having a coating (e.g., a coating to block radiation, such as Teflon) on the bulb to block radiation emissions from certain sections of the bulb. The coating may be utilized to block radiation emission from sections of the bulb coincident specific slots 89 of end-cap 72 to control radiation intensity and ozone production as described above.

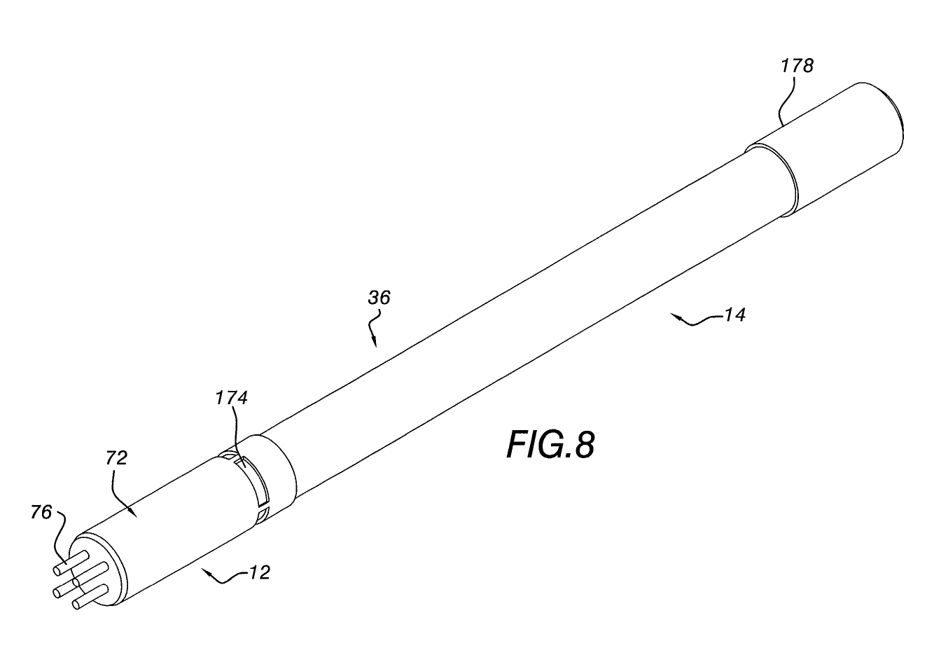

Shielding of ozone section 12 may be further accomplished via an end-cap 72 having windows for regulating emission of ozone generating radiation from radiation source 36 as illustrated in FIG. 8. Specifically, radiation source 36 includes ozone section 12 and germicidal section 14 as described above, ozone regulating end-cap 72 and a germicidal endcap 178. Radiation source 36 is typically disposed within a system with ozone and germicidal sections 12, 14 respectively disposed in the ozone and germicidal chambers as described above. Germicidal end-cap 178 is substantially cylindrical and typically includes an open bottom portion with cross-sectional dimensions greater than the cross-sectional dimensions of radiation source 36 to receive the end of the radiation source adjacent germicidal section 14. End-cap 178 covers a slight portion of germicidal section 14, and may be of any size or shape.

Ozone regulating end-cap 72 is substantially cylindrical and includes an open top portion with cross-sectional dimensions greater than the cross-sectional dimensions of radiation source 36 to receive the end of the radiation source adjacent ozone section 12. End-cap 72 may cover any portion of ozone section 12, may be of any size or shape and may be constructed of any suitable materials, such as plastic, that block or prevent passage of radiation from the ozone section. A series of openings or windows 174 are defined in end-cap 72 to regulate the amount of ozone generating radiation emitted in the ozone chamber (e.g., the amount of ozone generating radiation permitted to pass from the radiation source through the end-cap into the ozone chamber), thereby controlling ozone production. Windows 174 are substantially rectangular and are generally defined within end-cap 72 toward an end-cap upper portion. The windows are arranged about the end-cap exterior surface in a non-overlapping manner angularly spaced a slight distance from each other, and may include a glass or other radiation transparent covering. By way of example only, end-cap 72 includes four windows each having a width (e.g., transverse) or shorter rectangular dimension of approximately one-quarter of an inch. The remaining portions of end-cap 72 block radiation, thereby enabling windows 174 to regulate the amount of ozone generating radiation present within the ozone chamber and the quantity of ozone produced. Windows 174 may be of any size or shape, and may be disposed in any quantity (e.g., at least one) and in any fashion about end-cap 72 to facilitate emission of particular radiation intensities within the ozone chamber to produce desired ozone concentrations.

In addition, end-cap 72 includes pins or prongs 76 that extend distally from the end-cap distal end to enable radiation source 36 to receive power from a ballast (not shown) within the system. The pins are typically substantially cylindrical, but may be of any shape or size and may be constructed of any suitable materials. The pins are generally implemented by any type of conventional pins that enable connection to a connector or power source. By way of example only, end-cap 72 includes four pins arranged in a box-like configuration of two rows and two columns, however, the end-cap may include any quantity (e.g., at least one) of pins arranged on the end-cap in any fashion. Alternatively, end-cap 72 may include a ballast to directly provide power to the radiation source from the end-cap, and may be configured to implement the guiding mechanisms described above for FIGS. 4-5.

Ozone production may alternatively be controlled by disposing a sleeve about ozone section 12 to regulate radiation intensity within ozone chamber 8. In particular, the sleeve is constructed of material that blocks radiation and may be manipulable by gears or other mechanical and/or electrical devices to slide along and cover various portions of ozone section 12. The sleeve may be of any shape or size, and may include a fixed length to slide along and cover a specific portion of the ozone section. Alternatively, the sleeve may be collapsible or compressed to expand and contract along the ozone section to selectively cover specific portions or areas of the ozone section to control radiation intensity within the ozone chamber. Further, ozone production may similarly be controlled via a sleeve in substantially the same manner described above by regulating emission of germicidal radiation within the germicidal chamber since germicidal radiation removes ozone from the air stream as described above. The sleeve may be manipulable along the entire radiation source to control emission of ozone generating and/or germicidal radiation emitted from the radiation source depending upon the position of the sleeve along the source. Moreover, the radiation source may be coated (e.g., with Teflon) in a particular fashion to control emission of ozone generating and/or germicidal radiation from the radiation source. The coating blocks radiation emission, whereby the ozone and/or germicidal sections of the radiation source may be coated in any fashion to achieve a desired radiation intensity. The coating may be utilized to control ozone production by blocking radiation emitted from the radiation source in a manner similar to that described above for the sleeve.

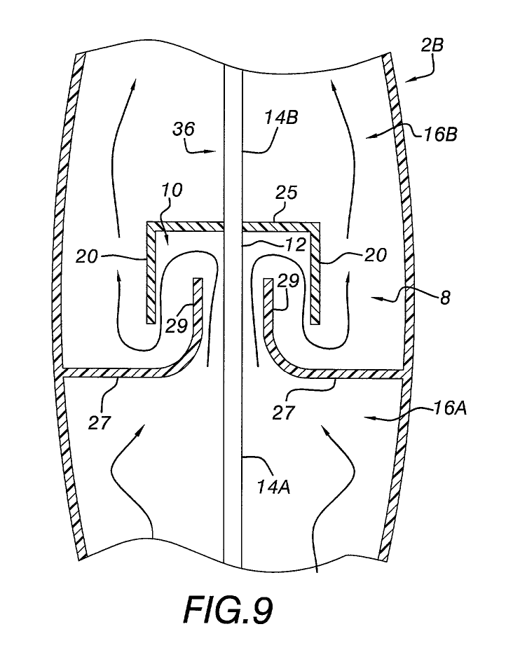

System 2a may include various configurations to reduce air through-flow velocity and enhance distribution of ozone within the air stream. For example, the ozone and germicidal chambers may include various winding, vortical or helical paths for the air stream to traverse, or the ozone chamber may include a vortex chamber to control air flow as described in the aforementioned patent applications. In addition, system 2a may be configured to include an additional germicidal chamber as illustrated in FIG. 9. Specifically, system 2b is substantially similar to and functions in a similar manner as system 2a described above except that system 2b includes an additional germicidal chamber to remove contaminants from an air stream prior to the air stream traversing an ozone chamber. System 2b includes ozone chamber 8 and germicidal chambers 16a, 16b wherein the ozone chamber is disposed between the germicidal chambers. The ozone and germicidal chambers are substantially similar to and function in substantially the same manner as the ozone and germicidal chambers described above. Radiation source 36 is similar to the radiation source described above and includes ozone section 12 emitting radiation having a wavelength of approximately 185 nanometers, and germicidal sections 14a, 14b that each emit radiation having a wavelength of approximately 254 nanometers as described above. The radiation source may be configured such that ozone section 12 and germicidal sections 14a, 14b each emit radiation at a high intensity or each section emits radiation at a low intensity, or ozone section 12 emits radiation at a low intensity, while germicidal sections 14a, 14b emit radiation at a high intensity. However, the radiation source may be configured for any desired radiation intensity emission, whereby the radiation intensities may be controlled by coating sections of the radiation source or any other techniques described above. Radiation source 36 is disposed within system 2b such that ozone section 12 and germicidal sections 14a, 14b reside within ozone chamber 8 and germicidal chambers 16a, 16b, respectively. Air enters system 2b via an intake (not shown) as described above and is directed into germicidal chamber 16a. Germicidal chamber 16a exposes the air stream to germicidal radiation emitted by germicidal section 14a to remove contaminants from the air stream as described above. The air stream subsequently enters ozone chamber 8 via an opening defined in the intermediate portion of lower ozone divider 27 as described above.

Ozone chamber 8 receives the air stream from germicidal chamber 16a and exposes the air stream to radiation emitted from ozone section 12 to produce ozone. Path 10 is formed within ozone chamber 8 via upper and lower ozone dividers 25, 27 and walls 20, 29 as described above to reduce air through-flow velocity and permit generated ozone to mix and interact with the air stream to remove contaminants. Since contaminants are initially removed from the air stream within germicidal chamber 16a prior to traversing ozone chamber 8 as described above, lesser quantities of contaminants reside within the air stream, thereby reducing the quantity of ozone needed to purify the air. Thus, ozone chamber 8 includes dimensions less than the dimensions of the ozone chamber described above, while ozone section 12 encompasses a smaller portion of radiation source 36 than the ozone section described above in order to produce reduced quantities of ozone for removal of the residual contaminants from the air stream. The air stream traverses path 10 wherein ozone generated from radiation emitted by ozone section 12 interacts and mixes with the air stream to remove contaminants as described above.

After traversing path 10, the air stream enters germicidal chamber 16b via gaps between upper divider 25 and the system housing as described above. The germicidal chamber exposes the air stream to germicidal radiation emitted from germicidal section 14b to remove contaminants and ozone from the air stream to produce sterilized air. Thus, the system sterilizes air with reduced quantities of ozone, thereby enhancing removal of ozone from the air stream. System 2b may include any quantity of chambers arranged in any fashion with radiation source 36 including any quantity of sections emitting radiation at specific wavelengths.

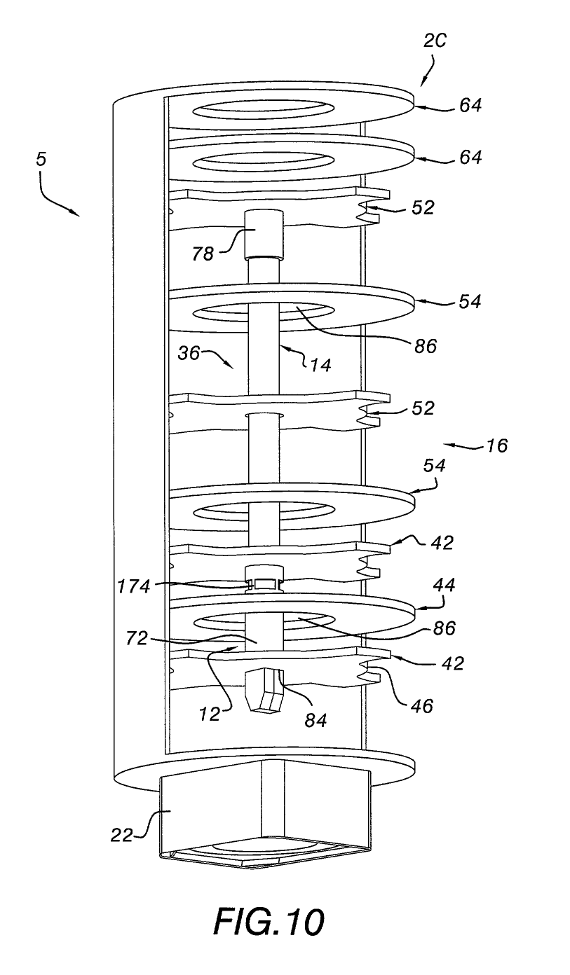

System 2a described above may include various configurations to reduce air through-flow velocity and enhance distribution of ozone within the air stream. An exemplary embodiment of the system described above having an alternative configuration to reduce air through-flow velocity and enhance distribution of ozone within the air stream is illustrated in FIG. 10. Specifically, system 2c is similar to system 2a described above and includes a housing 5, ozone and germicidal chambers 8, 16, a combination radiation source 36 having an ozone section 12 and a germicidal section 14, and an internal fan 22. Fan 22 draws an air stream from a surrounding environment into the system and directs the air stream into ozone chamber 8. Ozone chamber 8 is disposed adjacent fan 22 and includes ozone section 12 of radiation source 36 and a serpentine or tortuous air flow path formed by a plurality of baffles 42, 44 to enhance distribution of ozone within the air stream. Ozone section 12 typically is covered by end-cap 72 described above to regulate emission of ozone generating radiation within the ozone chamber and the amount of ozone produced. The serpentine path within ozone chamber 8 is generally formed by three baffles (e.g., baffle 44 disposed between a pair of baffles 42), however, the path may be formed by any quantity (e.g., at least one) of baffles disposed within the ozone chamber in any fashion. Windows 174 of end-cap 72 are preferably disposed in the ozone chamber between two baffles positioned toward germicidal section 14.

Two types of baffles are generally employed to form the air flow path. In particular, baffle 42 is substantially annular and includes an opening 84 defined toward the baffle center a and a plurality of recesses or cut-out portions 46 disposed about the baffle peripheral edge. The baffle opening includes dimensions slightly greater than the cross-sectional dimensions of radiation source 36 to receive the radiation source. By way of example only, baffle 42 includes a cross-sectional dimension between non-recessed baffle portions of approximately five inches, and a cross-sectional dimension between baffle recesses 46 of approximately four inches. Thus, each baffle recess 46 extends from a peripheral baffle edge toward the baffle center for approximately one-half inch. Baffle 44 is substantially annular and includes an opening 86 defined toward the baffle center, whereby the opening generally includes dimensions substantially greater than the cross-sectional dimensions of the radiation source. By way of example only, baffle 44 includes a cross-sectional diameter of approximately five inches, while the baffle opening includes a cross-sectional diameter of approximately three inches. However, openings 84, 86 may be of any suitable size or shape. The substantially central openings 84, 86 defined in baffles 42, 44 receive radiation source 36, while an air stream alternately flows through recesses 46 of baffles 42 and substantially central opening 86 of baffle 44 to traverse the ozone chamber in a serpentine or tortuous manner. By way of example only, the distance between the first and third baffle (e.g., baffles 42) within ozone chamber 8 is approximately two inches. Radiation emitted through windows 174 spreads throughout the ozone chamber, thereby irradiating the air stream prior to the air stream entering the germicidal chamber. In effect, baffles 42, 44 enlarge the ozone chamber by directing the air stream in a serpentine manner, thereby lengthening the ozone chamber path and creating turbulence to mix the ozone with the air stream.

Germicidal chamber 16 is disposed adjacent ozone chamber 8 and similarly includes a series of baffles 52, 54. Germicidal chamber baffles 52, 54 are disposed in an alternating fashion within the germicidal chamber and are typically separated by a distance greater than the separation distance of the baffles in the ozone chamber. Baffle 52 is substantially similar to baffle 42 described above, while baffle 54 is substantially similar to baffle 44 described above. The air stream flows in a serpentine manner through germicidal chamber baffles 52, 54 in substantially the same manner described above for ozone chamber baffles 42, 44, while being exposed to germicidal radiation to remove residual contaminants and ozone from the air stream. The air flow path through the germicidal chamber is typically formed by four baffles (e.g., two each of baffles 52, 54 alternately disposed preferably with baffle 54 initiating the baffle arrangement), however, the path may be formed by any quantity of baffles disposed within the germicidal chamber in any fashion. Additional baffles 64 are disposed beyond the radiation source (e.g., the radiation source length is less than the length of housing 5) between germicidal chamber 16 and a system exhaust in order to enable baffles 64 to maintain the emitted radiation within the system. Baffles 64 are substantially similar to baffles 44, 54 described above, whereby the system generally includes two baffles 64 to maintain emitted radiation within the system. However, the system may include any quantity (e.g., at least one) of baffles 64 to handle the emitted radiation. It is to be understood that baffles 42, 44, 52, 54 and 64 may be of any shape or size, may be configured in any manner and may be constructed of any suitable materials to direct air flow in a tortuous manner through housing 5.

Air flow through system 2c is described. Specifically, air enters system 2c via an air intake (not shown) and is directed by fan 22 into ozone chamber 8. The air stream traverses the serpentine path formed by baffles 42, 44 described above, whereby the air stream is exposed to ozone generating radiation emitted through end-cap windows 174 from ozone section 12 of radiation source 36. The ozone generating radiation produces ozone within the air stream, while the serpentine path formed by baffles 42, 44 enables the ozone to mix and interact with the air steam to remove contaminants. The air stream subsequently enters germicidal chamber 16 and traverses the serpentine path formed by germicidal chamber baffles 52, 54 described above. The air stream is exposed to germicidal radiation from germicidal section 14 to remove residual contaminants and ozone from the air stream. Subsequent to the germicidal chamber, the sterilized air stream traverses additional baffles 64 and returns to the surrounding environment via a system exhaust.

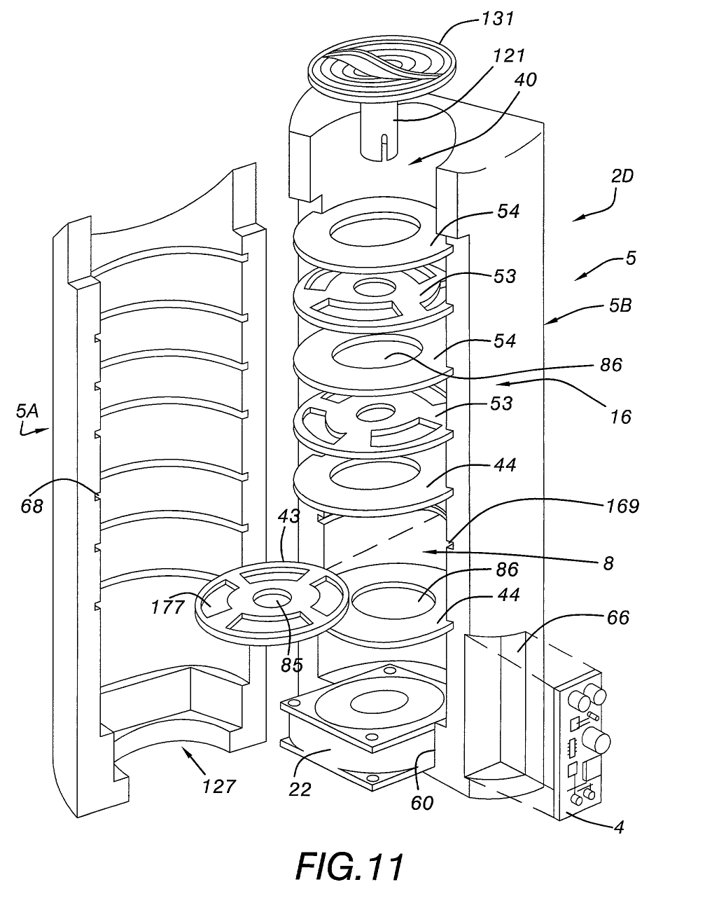

An alternative embodiment of the system of FIG. 10, especially for use as a wall unit, is illustrated in FIG. 11. Specifically, system 2d includes a housing 5, ozone and germicidal chambers 8, 16, a combination radiation source (not shown), an exhaust vent 131, a fan 22 and a ballast 4. Housing 5 is typically constructed of foam and includes front and rear components 5a, 5b that interface to form the system housing. Housing component 5a is generally semi-cylindrical having an open top portion and a partially closed bottom portion. substantially rectangular recess 127 is disposed toward the bottom of housing component 5a and includes a generally semi-circular opening defined in the recess floor. A series of slots 68 are further defined and longitudinally spaced apart in the interior surface of housing component 5a with each slot extending in the direction of a housing component transverse axis along an interior perimeter of that housing component. Housing component 5b is in the form of a generally trapezoidal block having a substantially semi-circular channel 40 defined in the block. The channel extends in the direction of a block longitudinal axis, thereby providing the block with partially open top and bottom portions. A series of slots 169, substantially similar to slots 68, are defined and longitudinally spaced apart in the interior surface of channel 40 and extend in the direction of a channel transverse axis along an interior channel perimeter. The bottom portion of housing component 5b includes a substantially rectangular recess 60 having a generally semi-circular opening defined in the recess floor. Further, a substantially rectangular recess 66 is disposed in a block side wall toward the bottom portion of housing component 5b to house ballast 4.

Housing components 5a, 5b interface to form a substantially cylindrical passageway to treat an air stream, while slots 68 and recess 127 of housing component 5a are respectively positioned coincident slots 169 and recess 60 of housing component 5b. Slots 68, 169 form receptacles to receive and secure baffles within the system that direct an air stream in a serpentine manner as described below. Recesses 60, 127 of the housing components form a substantially rectangular receptacle to receive fan 22, while the recess floor openings enable air to be drawn into and through the system by the fan. An external housing cover (not shown), typically constructed of plastic, is generally placed over housing 5.

Ozone chamber 8 is disposed adjacent fan 22 and includes baffles 43, 44 that form a serpentine air flow path through the ozone chamber in a similar manner as described above for system 2c. The path through the ozone chamber is typically formed by three baffles (e.g., baffle 43 disposed between a pair of baffles 44), however, the baffles may be arranged in any fashion and may be of any quantity (e.g., at least one). Baffle 43 is substantially annular and includes an opening 85 defined toward the baffle center having dimensions slightly greater than the cross-sectional dimensions of the radiation source. Further, baffle 43 includes openings 177 defined about an exterior surface of baffle 43 toward the baffle peripheral edges, whereby the openings are arranged in a non-overlapping manner angularly spaced a slight distance from each other. Openings 177 are generally rectangular having curved edges along their longer rectangular dimension, however, the openings may be of any shape, size or quantity (e.g., at least one). The radiation source is substantially similar to radiation source 36 described above and is typically positioned such that ozone section 12 is disposed through the openings defined toward the centers of baffles 43, 44 as described above. Baffle 44 is substantially annular and includes an opening 86 substantially greater than the cross-sectional dimensions of the radiation source as described above. Air flows within the ozone chamber through opening 86 of baffle 44 and openings 177 of baffle 43, whereby baffles 44 direct air inward toward the radiation source, while openings 177 direct air outward toward passageway walls to form a serpentine air flow path through the ozone chamber. An air stream is directed into the ozone chamber via fan 22, whereby the air is exposed to ozone generating radiation as described above. The serpentine path formed by baffles 43, 44 enables generated ozone to mix and interact with the air stream to remove contaminants.

Germicidal chamber 16 is disposed adjacent ozone chamber 8 and similarly includes baffles 53, 54 alternately arranged to form a serpentine path through the germicidal chamber in substantially the same manner described above. The germicidal chamber typically includes four baffles (e.g., two each of baffles 53, 54 alternately disposed preferably with baffle 53 initiating the baffle arrangement), however, the baffles may be arranged in any fashion and may be of any quantity (e.g., at least one). Baffle 53 is substantially similar to baffle 43 described above, while baffle 54 is substantially similar to baffle 44 described above. The air stream enters the germicidal chamber from ozone chamber 8, whereby the air stream traverses the serpentine path formed by baffles 53, 54 and is exposed to germicidal radiation from the radiation source germicidal section to remove residual contaminants and ozone from the air stream. Sterilized air exits the germicidal chamber and returns to the surrounding environment via exhaust vent 131. Exhaust vent 131 is typically substantially circular and includes a bulb holder 121 extending from the vent into the system to engage an end of the radiation source adjacent the germicidal section. Bulb holder 121 is generally cylindrical having cross-sectional dimensions slightly larger then the cross-sectional dimensions of the radiation source to receive the radiation source end. The exhaust and bulb holder vent permit placement and removal of the radiation source within the system and may be of any shape or size.

Air flow through system 2d is described. The air flow path is substantially similar to the air flow path described above for system 2c (FIG. 10). Specifically, air enters the system via an air intake (not shown) and is directed into ozone chamber 8 by fan 22. The air stream traverses the alternating sequence of openings 86 of baffles 44 and openings 177 of baffle 43 to flow in a serpentine manner through the ozone chamber. Ozone generating radiation is emitted by the radiation source (not shown) to generate ozone within the air stream. The serpentine path enables the ozone to mix and interact with the air steam to permit the ozone to remove contaminants. The air and ozone mixture enters germicidal chamber 16 from the ozone chamber and traverses the alternating sequence of openings 86 of germicidal baffles 54 and openings 177 of baffles 53 to flow in a serpentine fashion through the germicidal chamber as described above. The air stream is exposed to germicidal radiation to remove residual contaminants and ozone from the air stream to produce sterilized air, whereby the sterilized air flows through exhaust vent 131 to return to the surrounding environment.

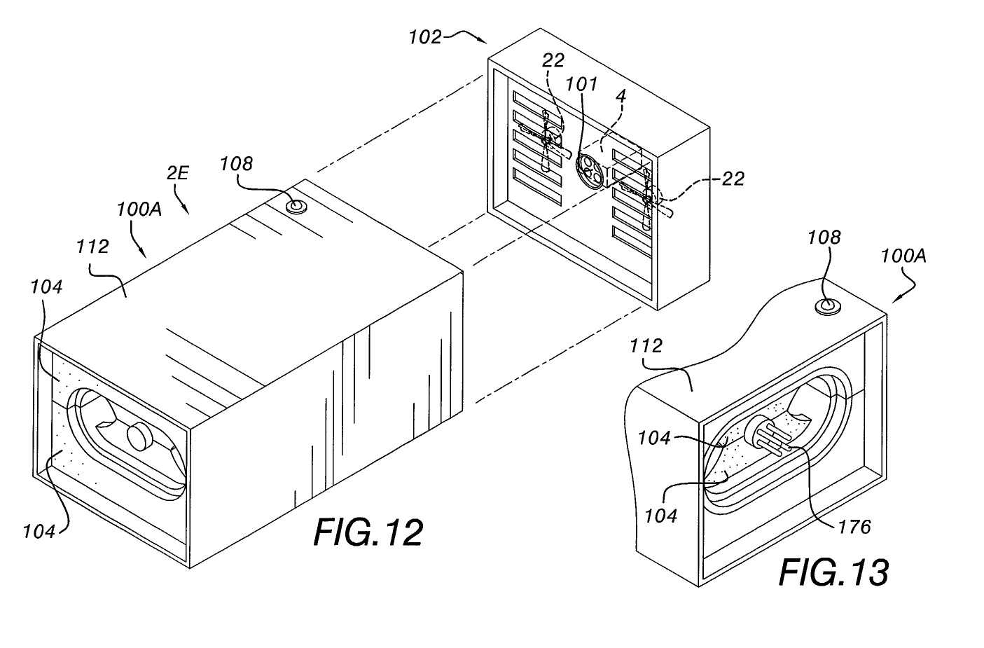

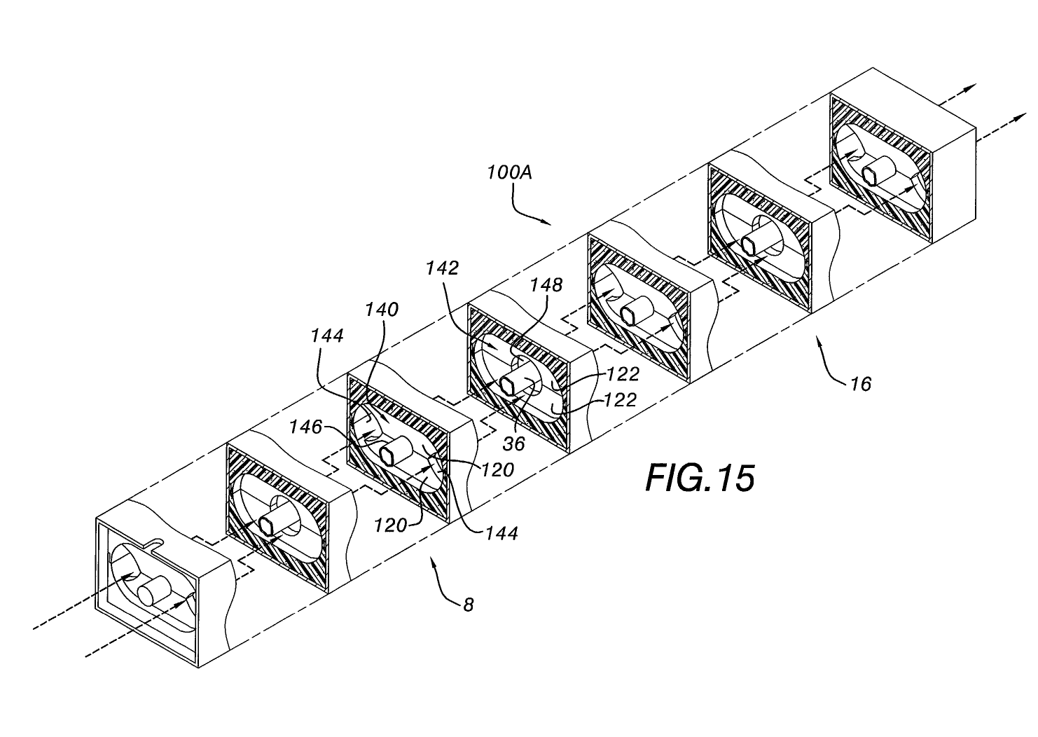

A system employing a replaceable cartridge for producing purified or ozone enriched air is illustrated in FIGS. 12-13. Specifically, system 2e is similar to systems 2a, 2c-2d described above and includes a base 102 for housing system electrical components (e.g., ballast, fan, etc.) and a cartridge 100a having ozone and germicidal chambers 8, 16 and radiation source 36 (FIG. 15). Base 102 is typically disposed in an area containing air to be treated, while cartridge 100a is connected to the base to treat the air in the surrounding environment. The cartridge is preferably disposable and may be replaced as needed, while the base receives and interacts with the replaceable cartridges to remove contaminants from an air stream. Base 102 is typically substantially rectangular and includes dimensions greater than the cross-sectional dimensions of the cartridge to enable the base to receive the cartridge. The base houses the electrical components for system 2e and includes fans 22 (e.g., at least one fan) to direct air from a surrounding environment into cartridge 100a, a ballast 4 to provide current to the radiation source, a power receptacle 101 for facilitating connections between the cartridge and power sources (e.g., ballast), and any other electrical components needed by the system. Ballast 4 may be an A.C. ballast, whereby base 102 is connected to an A.C. power source, such as a conventional wall outlet jack. Alternatively, base 102 may include a D.C. ballast and either be connected to a vehicle power system or have a battery for powering the ballast. The power receptacle typically includes a series of pin receptacles to receive elongated pins 176 from a radiation source end-cap. The power receptacle may be implemented by any conventional or other receptacle, whereby the pin receptacles may be of any quantity, shape or size, and may be arranged in any fashion. Further, the base components (e.g., ballasts, power receptacles, etc.) may be of any quantity (e.g., at least one) and may be arranged in any fashion capable of performing their desired functions. Moreover, the base and cartridge each may be of any size or shape, and may be disposed in any fashion capable of enabling the base to provide power to and direct air through the cartridge.

Cartridge 100a typically includes cartridge components 104 that interface to form the cartridge housing. Each cartridge component is configured to essentially implement half of the cartridge housing (e.g., two substantially identical cartridge components may be utilized to form the cartridge housing). The cartridge includes ozone and germicidal chambers and a radiation source, and is configured to direct air in a serpentine manner and to treat the air in substantially the same manner as the systems described above. The cartridge is typically constructed of plastic foam (e.g., polystyrene, expanded polypropylene foam, closed cell or packaging foam, heat seal foam, or foams from the group of polyvinyl aromatic hydrocarbons or any other foam), but may be constructed of any suitable materials. Further, the foam may be a combination of foams or treated with various liners or chemicals via vacuum metalizing or other techniques for handling of liquids, fire retardation or to increase foam capabilities (e.g., strength, tolerance to heat, cold, liquid, chemicals, etc.). An indicator 108, preferably a conventional light emitting diode (LED), is disposed on the cartridge toward the cartridge rear portion to indicate operation of the radiation source. The indicator generally receives power from receptacle 101 and monitors the radiation source. A sleeve 112 is typically disposed over and covers cartridge 100a, whereby the sleeve is preferably constructed of plastic, but may be constructed of any suitable materials.

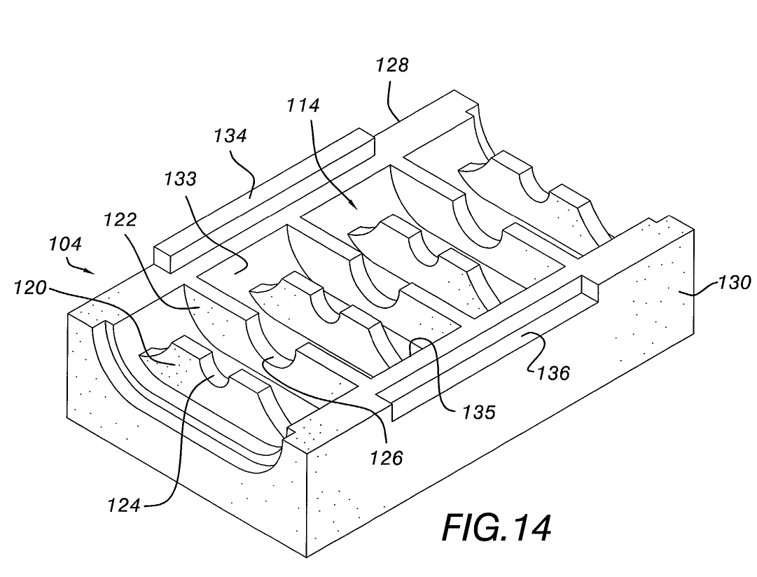

Cartridge component 104 for forming the cartridge housing is illustrated in FIG. 14. Specifically, cartridge component 104 is in the form of a rectangular block having side walls 128, 130 and a channel 114 extending in a direction of a block longitudinal axis. Channel 114 includes side walls 133, 135 and a series of walls 120, 122 alternately disposed and longitudinally separated by a slight distance within the channel. Wall 120 occupies the space between the bottom portions of channel side walls 133, 135 and extends from the channel floor toward the channel side wall upper edges. Wall 120 is configured with cut-away segments to form gaps between upper edge portions of wall 120 and the channel side walls to enable an air stream to traverse those gaps during treatment as described below. A generally semi-circular recess 124 is disposed toward the approximate center of the upper portion of wall 120 and extends from that upper portion inwardly toward the wall center. Recess 124 typically receives and secures the radiation source within the cartridge in close fitting relation.

Wall 122 is similarly disposed between the channel side walls and extends in a direction of a channel transverse axis along the interior channel perimeter. A generally semi-circular recess 126 is disposed proximate the center of the upper edge portion of wall 122 and extends inwardly from that upper edge portion toward the wall bottom. Recess 126 includes dimensions greater than the dimensions of recess 124 to permit air flow through recess 126 during treatment as described below. Channel 114 typically includes seven walls (e.g., four walls 120 and three walls 122 disposed in alternating fashion with each wall 122 disposed between a pair of walls 120), whereby the first three walls typically form ozone chamber 8, while the remaining walls generally form germicidal chamber 16. However, the ozone and germicidal chambers may each include any quantity of walls (e.g., at least one) arranged in any fashion. Block side walls 128, 130 are configured to enable cartridge components 104 to interlock, whereby a raised tab portion or step 134 is disposed toward the approximate longitudinal center of the upper edge of side wall 128, while a corresponding recess 136 is disposed toward the approximate longitudinal center of the upper edge of side wall 130. The raised tab portion and recess include substantially the same dimensions such that the tab of one cartridge component snugly fits into the recess of another cartridge component to interlock the cartridge components and form the cartridge housing. However, the block may include any fastening devices or techniques to enable cartridge components to interlock.

When cartridge components interface, the components form the internal structure of ozone and germicidal chambers 8, 16 to remove contaminants from an air stream as illustrated in FIG. 15. Specifically, cartridge 100a is formed by two identical interlocking cartridge components 104 and includes ozone chamber 8 and germicidal chamber 16. The cartridge components interface as described above, whereby edges of walls 120, 122 of each cartridge component are positioned coincident each other to respectively form walls 140, 142 that direct air flow through the cartridge. Wall 140 includes an opening 146 defined toward the approximate center of wall 140. Opening 146 is formed by recesses 124 of coincident edges of walls 120 and includes dimensions only slightly greater than or equal to the cross-sectional dimensions of radiation source 36 to receive ozone section 12 of the source. Openings 144 are defined in wall 140 toward the cartridge side walls to direct an air stream away from the radiation source as the air stream traverses the ozone chamber. Wall 142 includes an opening 148 defined toward the approximate center of wall 142. Opening 148 is formed by recesses 126 of coincident edges of walls 122 and includes dimensions substantially greater than the cross-sectional dimensions of radiation source 36 to direct the air stream toward the radiation source as the air stream traverses the ozone chamber. The sequence of walls 140, 142 within the ozone chamber directs the air stream to alternately flow with an outward flow component toward the cartridge side walls and then with an inward flow component toward the radiation source, thereby directing the air stream through the ozone chamber in a generally three-dimensional serpentine manner. Air entering the ozone chamber is exposed to ozone generating radiation that produces ozone within the air stream, whereby walls 140, 142 direct the air stream in a serpentine fashion to mix the ozone with the air stream to remove contaminants. Ozone chamber 8 typically includes three walls (e.g., wall 142 disposed between a pair of walls 140), however, the ozone chamber may include any quantity (e.g., at least one) of walls arranged in any fashion.

Germicidal chamber 16 is disposed adjacent ozone chamber 8, whereby openings 146, 148 receive and secure germicidal section 14 within the germicidal chamber. Air enters germicidal chamber 16 from ozone chamber 8 and is exposed to germicidal radiation to remove residual contaminants and ozone residing within the air stream. Openings 148 of walls 142 and openings 144 of walls 140 direct the air stream to flow in a serpentine manner through the germicidal chamber in substantially the same manner described above, whereby sterilized air from the germicidal chamber returns to a surrounding environment via a system exhaust (not shown). Germicidal chamber 16 typically includes four walls (e.g., two each of walls 140, 142 disposed in an alternating fashion preferably with wall 142 initiating the arrangement), however, the germicidal chamber may include any quantity (e.g., at least one) of walls arranged in any fashion.

Operation of the system is described with reference to FIGS. 12-13 and 15. Initially, base 102 is disposed in an appropriate location (e.g., room, vehicle, duct system, etc.). Cartridge 100a including radiation source 36 is connected to base 102 via receptacle 101 to provide power to the cartridge and direct air through the system. An air stream from a surrounding environment is directed into the system, via fan 22, and enters ozone chamber 8. The air stream is exposed to ozone generating radiation and traverses a serpentine air flow path formed by openings in walls 140, 142 as described above. The serpentine air flow path enables the ozone to mix and interact with the air stream to remove contaminants. The air stream subsequently enters germicidal chamber 16 wherein the air stream is exposed to germicidal radiation to remove residual contaminants and ozone residing within that air stream. The air stream traverses the serpentine air flow path within the germicidal chamber formed by openings in walls 140, 142 and exits the system via a system exhaust. The base and cartridge may be of any shape or size to accommodate any sized areas or various applications.

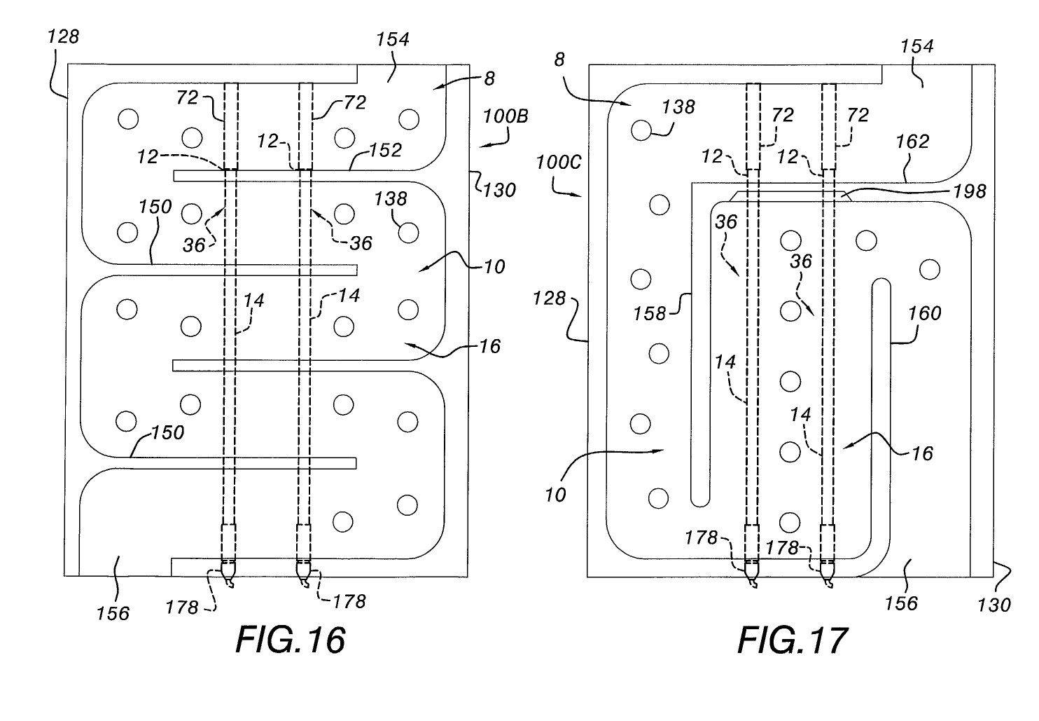

The cartridge described above may include various configurations to produce a serpentine air flow path and reduce through-flow velocity in the ozone chamber. An exemplary configuration for the cartridge to provide a serpentine air flow path is illustrated in FIG. 16. Specifically, cartridge 100b includes ozone chamber 8, germicidal chamber 16 and a pair of combination radiation sources 36 each having an ozone section 12 and germicidal section 14 as described above. However, cartridge 100b may include any quantity (e.g., at least one) of radiation sources. Cartridge side wall 128 (e.g., the leftmost side wall as viewed in FIG. 16) includes dividers 150 extending from that side wall toward side wall 130 (e.g., the rightmost side wall as viewed in FIG. 16), while side wall 130 includes dividers 152 extending from that side wall toward side wall 128. Dividers 150 each extend from side wall 128 for a distance slightly less than the distance between side walls 128, 130, thereby forming respective gaps between dividers 150 and side wall 130. Similarly, dividers 152 each extend from side wall 130 for a distance slightly less than the distance between side walls 128, 130, thereby forming respective gaps between dividers 152 and side wall 128. Dividers 150, 152 are interleaved to form successive passageways that collectively define a serpentine path 10 through the cartridge. A plurality of posts 138 are disposed along path 10 to reduce air through-flow velocity and generate turbulence within the flowing air.

Radiation sources 36 extend in the direction of a longitudinal axis of the cartridge and are disposed toward the approximate center of the front and rear cartridge walls. Ozone chamber 8 generally occupies the portion of serpentine path 10 residing between a cartridge front wall and a divider 152 positioned closest to the front wall. An end-cap 72 is disposed over ozone section 12 of each radiation source, and includes windows (not shown) to regulate emission of ozone generating radiation and production of ozone. End-caps 72 interface base 102 (e.g., with plural ballasts) to supply power to the cartridge. Air enters ozone chamber 8 via an intake 154 defined in the cartridge front wall toward side wall 130, and is exposed to ozone generating radiation from ozone section 12 to produce ozone. The air stream traverses posts 138, disposed within the ozone chamber toward side wall 128, to reduce air through-flow velocity and enable the ozone to mix with the air.

Germicidal chamber 16 effectively occupies the remaining portion of path 10 and similarly includes posts 138 or other forms of obstruction to reduce air through-flow velocity and generate turbulence in the flowing air. The air stream is exposed to germicidal radiation from germicidal section 14 of radiation source 36 to remove residual contaminants and ozone as the air stream traverses the path within the germicidal chamber. The air stream exits the 131 system and returns to the surrounding environment via an exhaust 156 defined in the cartridge rear wall toward side wall 128. Each radiation source 36 includes a germicidal end-cap 178 that receives an end of the radiation source adjacent its germicidal section to secure the radiation source in the cartridge.

Air flow through cartridge 100b is described. Specifically, an air stream enters cartridge 100b via intake 154 and is directed into ozone chamber 8. The air stream is exposed to ozone generating radiation from ozone section 12 of each radiation source and produces ozone to remove contaminants. The air stream traverses path 10 and posts 138 that enable the ozone to efficiently mix and interact with the air stream to remove contaminants. The air stream flows through path 10 and enters germicidal chamber 16 where the air stream is exposed to germicidal radiation from germicidal section 14 of each radiation source to remove residual contaminants and ozone. The air stream traverses path 10 and posts 138 and exits the system to the surrounding environment via exhaust 156.

An alternative configuration for the cartridge is illustrated in FIG. 17. Specifically, cartridge 100c includes ozone chamber 8, germicidal chamber 16, and a pair of combination radiation sources 36 each having an ozone section 12 and germicidal section 14 as described above. However, cartridge 100c may include any quantity (e.g., at least one) of radiation sources. Cartridge side wall 130 (e.g., the rightmost side wall as viewed in FIG. 17) includes a divider 162 extending from that side wall toward side wall 128 (e.g., the leftmost side wall as viewed in FIG. 17). Divider 162 extends from side wall 130 for a distance slightly less than the distance between side walls 128, 130 to form a gap between divider 162 and side wall 128. A divider 158 extends from divider 162 toward the cartridge rear wall substantially in parallel to side wall 128. Divider 158 has a length slightly less than the distance between divider 162 and the cartridge rear wall to form a gap between divider 158 and the cartridge rear wall. A divider 160 extends from the cartridge rear wall toward divider 162, and includes a length slightly less than the distance between the cartridge rear wall and divider 162 to form a gap between divider 160 and divider 162. The dividers form passageways through the cartridge that collectively define serpentine path 10. A plurality of posts 138 are disposed within path 10 to reduce air through-flow velocity and generate turbulence in the flowing air as described above.

Radiation sources 36 extend in the direction of a longitudinal axis of the cartridge and are disposed between dividers 158, 160 toward the approximate center between side walls 128, 130. Ozone chamber 8 occupies the portion of path 10 between the cartridge front wall and divider 162. An end-cap 72 is disposed over ozone section 12 of each radiation source, and includes windows (not shown) to regulate emission of ozone generating radiation and production of ozone. End-caps 72 interface base 102 (e.g., with plural ballasts) to supply power to the cartridge. Air enters ozone chamber 8 via an intake 154 defined in the cartridge front wall toward side wall 130, and is exposed to ozone generating radiation from ozone a section 12 to produce ozone. The air stream traverses posts 138 disposed along path 10 toward side wall 128 to reduce air through-flow velocity and enable the ozone to mix with the air. The portion of path 10 between divider 158 and side wall 128 essentially serves as a dwell time chamber to enable the ozone to mix and interact with the air.

Germicidal chamber 16 effectively occupies the remaining portions of path 10 subsequent to the dwell time chamber (e.g., the portions of path 10 between dividers 158 and 160 and between side wall 130 and divider 160). In other words, the germicidal chamber occupies the portions of path 10 capable of receiving germicidal radiation from germicidal section 14 of each radiation source. The germicidal chamber similarly includes posts 138 to reduce air through-flow velocity and generate turbulence in the air. The air stream is exposed to germicidal radiation from germicidal section 14 of each radiation source to remove residual contaminants and ozone as the air stream traverses the path within the germicidal chamber. Further, a conventional or other type of filter 198 may be disposed adjacent divider 162 to remove particulate or other contaminants from the air stream during traversal of the path. The air stream exits the system and returns to the surrounding environment via exhaust 156 defined in the cartridge rear wall toward side wall 130. Radiation sources 36 each include a germicidal end-cap 178 that receives an end of a corresponding radiation source adjacent germicidal section 14 to secure that radiation source within the cartridge.

Air flow through cartridge 100c is described. Specifically, an air stream enters cartridge 100c via intake 154 and is directed into ozone chamber 8. The air stream is exposed to ozone generating radiation from ozone section 12 of each radiation source and produces ozone to remove contaminants. The air stream traverses the dwell chamber within path 10 and posts 138 that enable the ozone to mix and interact with the air stream to remove contaminants. The air stream flows through path 10 and enters germicidal chamber 16 where the air stream is exposed to germicidal radiation from germicidal section 14 of each radiation source. The germicidal radiation removes residual contaminants and ozone from the air stream. The air stream traverses path 10 and posts 138 within the germicidal chamber, and exits the system to the surrounding environment via exhaust 156.

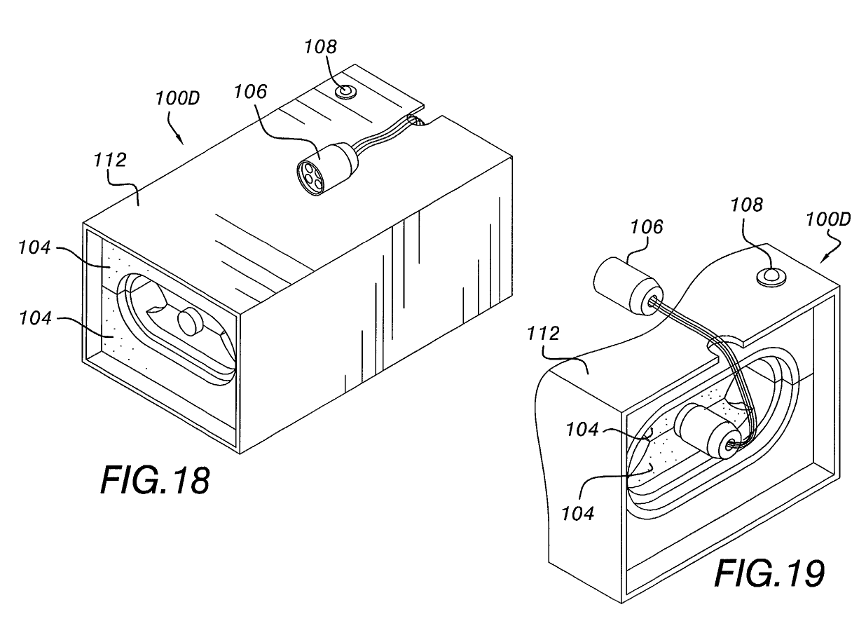

A cartridge configured for use within plenums of vehicles or other locations (e.g., ducts of HVAC systems) is illustrated in FIGS. 18-19. Specifically, cartridge 100d is substantially similar to and functions in substantially the same manner as the cartridges described above except that the cartridge includes a connector 106 to provide power to the cartridge. Cartridge 100d may include any of the cartridge configurations described above (e.g., with plural connectors for FIGS. 16-17), and is typically inserted within a plenum of a vehicle. Connector 106 interfaces a radiation source end-cap and extends to connect the cartridge to a vehicle or other power supply. The connector may be implemented by any conventional or other type of connector, and may be configured in any fashion (e.g., to handle multiple radiation sources) to facilitate connection between the cartridge and power source. Light indicator 108 may receive power from the power supply via connector 106.

Cartridge 100d is typically inserted within the plenum such that air flowing within the plenum directly flows through the cartridge. A fan may be disposed on the cartridge to assist in directing air through the cartridge, however, plenum air flow is generally sufficient to enable treatment of the air stream by the cartridge. An air stream enters the cartridge, whereby the air stream traverses the cartridge ozone and germicidal chambers to facilitate removal of contaminants from the air stream in substantially the same manner described above. Purified air may then be returned to the vehicle interior or other surrounding environment.

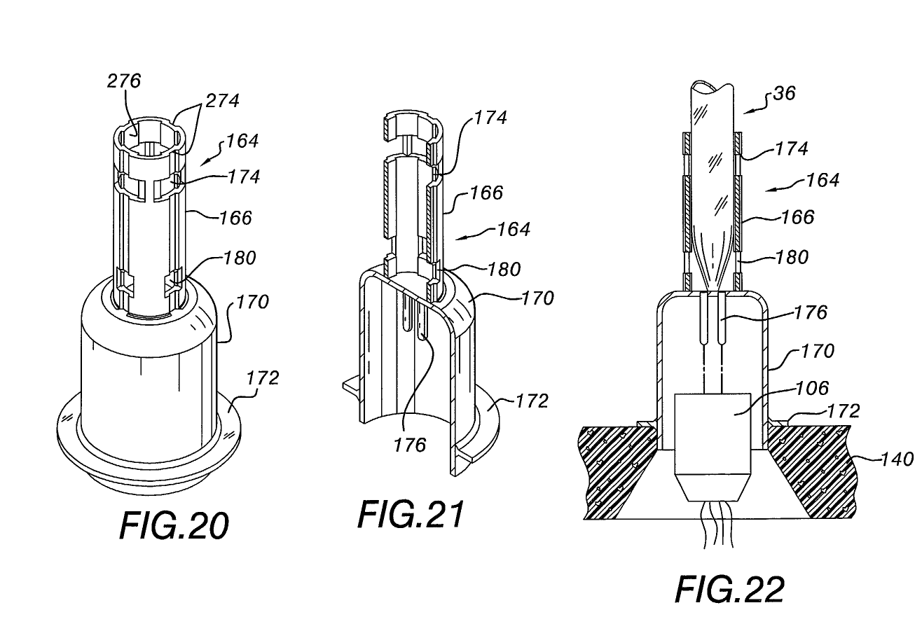

A radiation source end-cap for use with a cartridge to insulate the cartridge from radiation source temperatures is illustrated in FIGS. 20-21. Specifically, end-cap 164 includes a bulb receptacle 166, a support 170 and a flange 172. Bulb receptacle 166 is generally cylindrical having an open top portion to receive an end of a radiation source. The bulb receptacle includes a series of grooves 274 defined in the receptacle exterior surface and extending in the direction of a receptacle longitudinal axis. Similarly, a series of ridges 276 are defined in the interior surface of bulb receptacle 166 coincident grooves 274 and extend in the direction of a receptacle longitudinal axis. The bulb receptacle includes cross-sectional dimensions substantially similar to the cross-sectional dimensions of the radiation source such that ridges 276 extend from the receptacle interior surface to snugly receive an end of the radiation source. The bulb receptacle typically covers the ozone section and includes air vents 180 to permit cooling of the radiation source. The bulb receptacle may be constructed of any suitable materials capable of blocking radiation, and includes windows 174 as described above to regulate emission of ozone generating radiation and production of ozone. By way of example only, bulb receptacle 166 has a height of approximately one and three-quarters inches with an inner cross-sectional dimension of slightly greater than one-half inch.

Bulb receptacle 166 is disposed toward the approximate center of a top surface of support 170, whereby pins 176 extend from the distal end of receptacle 166 into the support interior to facilitate power connections for the radiation source. The receptacle generally includes four pins typically arranged in a box-like configuration of two rows and two columns with the pin rows separated by a distance of approximately 0.3 inches, however, the receptacle may include any quantity of pins arranged in any fashion. Support 170 is generally cylindrical, and includes an open bottom portion to enable access to the pins. The support has cross-sectional dimensions greater than the cross-sectional dimensions of receptacle 166. The support top surface interfaces the support side surfaces in such a manner to form a rounded junction or intersection. Flange or ledge 172 is disposed toward the bottom of the support, and extends from and about the support exterior surface. By way of example only, the flange is disposed approximately one and one-half inches from the support top surface. Support 170 is typically inserted within a receptacle in a cartridge, whereby flange 172 secures the end-cap in place, while support 170 elevates or provides sufficient distance between the cartridge and portion of the radiation source having substantial temperatures.

Operation of a radiation source and end-cap is described with reference to FIG. 22. Initially, an end of a radiation source 36 is inserted into bulb receptacle 166 of end-cap 164. Support 170 is typically inserted within an opening formed in a cartridge toward the cartridge rear wall (e.g., opening 146 in wall 140 (FIG. 15)). Flange 172 serves as a stop to secure the radiation source in that opening. Connector 106 of cartridge 100d may be inserted into the open bottom portion of support 170 to interface pins 176 or, alternatively, pins 176 may be elongated to extend beyond the end-cap to interface receptacle 101 of base 102 (e.g., cartridge 100a) in order to provide power to the respective cartridges. Support 170 provides sufficient distance between the end of the radiation source and the cartridge housing, whereby the end of the radiation source typically incurs substantial temperatures. Essentially, the end-cap maintains heat generated by the radiation source away from the foam cartridge housing, while the air vents permit air to pass over and cool the extreme temperatures of the bulb. In other words, the end-cap raises the hot bulb away from the foam cartridge housing to prevent substantial temperatures of the radiation source from affecting the housing.

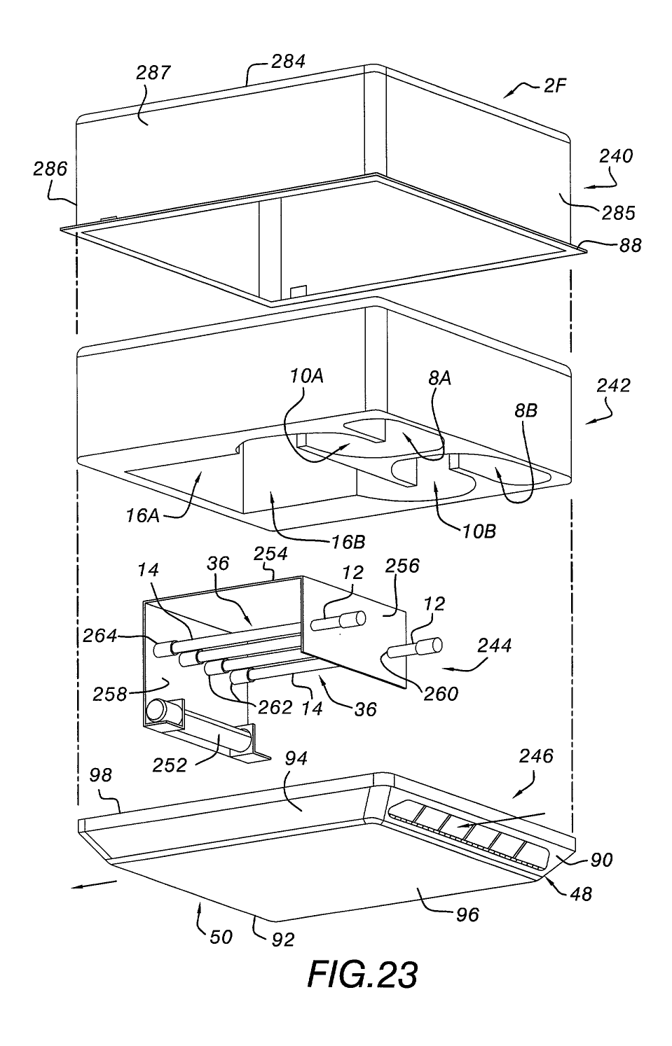

A system 2f for removing contaminants from an air stream to produce purified or ozone enriched air, typically for installation within a ceiling or wall, is illustrated in FIG. 23. System 2f is substantially similar to the corresponding ceiling or wall units disclosed in the aforementioned patent applications. Specifically, system 2f includes a cover or housing 240, chamber block 242, electrical component assembly 244, and a base 246. Base 246, typically constructed of molded plastic or other suitably sturdy material, includes substantially rectangular front, rear, side and bottom walls 90, 92, 94, 96, respectively, that collectively define a base interior. The bottom wall is substantially flat, while the front, rear and side walls are slightly tilted outward to expand the base interior. The upper portions of the front, rear and side walls are not tilted, but rather, extend in a substantially vertical fashion to form a base periphery 98. An intake vent 48 is disposed on base front wall 90, while an exhaust vent 50 is disposed on base rear wall 92. Base 246 may further include dividing walls (not shown) to prevent contact between the incoming contaminated air from intake vent 48 and the outgoing sterilized air to be exhausted through exhaust vent 50, and to distribute the incoming air stream from intake vent 48 to different ozone chambers as described below. A platform (not shown) is disposed slightly below base periphery 98 to cover and form an air chamber within the base interior. The platform is substantially rectangular and includes dimensions slightly less than the dimensions of periphery 98 to form gaps or openings between the platform and periphery adjacent the intake and exhaust vents. The openings enable incoming air to enter the system from intake vent 48, and enable outgoing air from the system to be exhausted through exhaust vent 50. The system may be inserted within a ceiling or wall such that only base 246 is visible within a room to enable the intake and exhaust vents to respectively receive and exhaust air to the room.

Chamber block 242 is typically a substantially rectangular block having cross-sectional dimensions slightly less than base 246 in order to be disposed on the base platform. Block 242 is typically constructed of expandable polypropylene close cell foam, a lightweight and sound and shock absorption material. However, chamber block 242 may be constructed of any other materials capable of forming ozone and germicidal chambers as described below. Chamber block 242 includes a pair of isolated ozone chambers 8a, 8b and a pair of germicidal chambers 16a, 16b, whereby each ozone and germicidal chamber is substantially similar to and functions in substantially the same manner as the respective corresponding ceiling or wall unit ozone and germicidal chambers described in the aforementioned patent applications. Specifically, ozone chambers 8a, 8b respectively include paths 10a, 10b formed into the foam block serving to reduce air through-flow velocity and enhance ozone distribution within the air stream as described above. The paths are each essentially defined by a winding groove or channel formed in the chamber block to reduce air through-flow velocity and mix generated ozone with the air stream to remove contaminants as described above. Paths 10a, 10b are each formed toward the front portion of the chamber block and extend toward the rear block portion into respective germicidal chambers 16a, 16b. Paths 10a, 10b tend to be mirror images of each other and direct air streams to enter the respective germicidal chambers.

Germicidal chambers 16a, 16b are formed in chamber block 242 adjacent respective ozone chambers 8a, 8b. The air streams from ozone chamber paths 10a, 10b enter the respective germicidal chambers from opposing sides of the chamber block. The germicidal chambers are collectively defined by a substantially rectangular recess formed in the chamber block wherein the germicidal chambers are typically not isolated, but rather, share a common area. Air streams from the ozone chambers are directed through the respective ozone chamber paths and enter germicidal chambers 16a, 16b or, in other words, the chamber block recess. The ozone and germicidal chambers each include radiation sources, whereby the radiation sources are disposed on electrical component assembly 244 for disposal within chamber block 242 as described below. The ozone and germicidal chambers may alternatively include any of the configurations described above to reduce air through-flow velocity and enable generated ozone to mix with the air as described above. The ozone generation and application of germicidal radiation may be controlled to produce ozone enriched air having a particular ozone concentration level for various applications as described below.

Electrical component assembly 244 is typically constructed of sheet metal or other suitably sturdy material and preferably includes two combination radiation sources 36 described above, two radiation sources 62 emitting germicidal radiation similar to germicidal section 14 of radiation source 36, fan 252 and other electrical components for the system, such as ballasts (not shown). The assembly typically includes a top wall 254, a front wall 256 and a rear wall 258. Each wall is substantially rectangular, whereby the front and rear walls respectively extend from the top wall front and rear edges substantially perpendicular to the top wall. Top wall 254 has dimensions slightly less than the dimensions of the recess within chamber block 242 forming the germicidal chambers such that assembly 244 is inserted within that recess. Rear wall 258 extends from top wall 254 for a distance substantially similar to the depth of the chamber block recess such that fan 252 is substantially flush with a recess peripheral edge when assembly 244 is disposed within the recess. Front wall 256 extends from top wall 254 substantially parallel to rear wall 258 for a distance slightly less than the extension of the rear wall. Front wall 256 includes an opening 260 disposed toward the approximate center of each front wall side edge, and a pair of receptacles 264 (not shown on front wall 256 in FIG. 23) disposed between openings 260. Similarly, rear wall 258 includes a receptacle 264 disposed coincident each opening 260 and receptacle 264 disposed on front wall 256. Openings 260 disposed on front wall 256 and their corresponding receptacles 264 disposed on rear wall 258 each receive a combination radiation source 36 such that the ozone section of the radiation source extends through opening 260 and is disposed external of the assembly, while germicidal section 14 remains within the assembly. Similarly, corresponding receptacles 264 disposed on the front and rear walls receive radiation sources 62. Receptacles 264 disposed on rear wall 258 typically include power connectors to provide current to the radiation sources from a ballast (not shown) via an end-cap described below. Fan 252 is attached to rear wall 258 below the radiation sources (e.g., as viewed in FIG. 23), and is typically implemented by a barrel or other type of fan or blower device to draw air through the system.

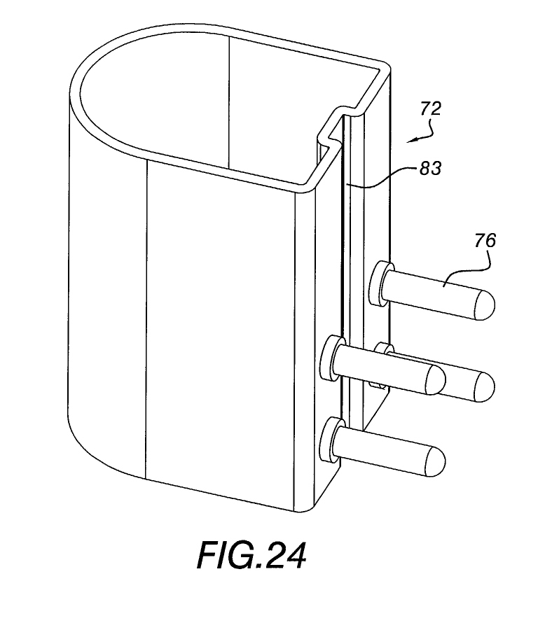

An end-cap for radiation sources 36, 62, enabling insertion of the radiation sources within connectors of receptacles 264, is illustrated, by way of example only, in FIG. 24. Specifically, end-cap 72 includes an open top portion and has dimensions slightly greater than the cross-sectional dimensions of radiation sources 36, 62 to receive an end of one of those radiation sources. The end-cap receives the end of radiation source 36 adjacent ozone section 14, or an end of radiation source 62 in its open top portion and includes a generally rectangular cross-section that tapers toward an end-cap far side (e.g., as viewed in FIG. 24) to form a rounded peak along the shorter rectangular cross-sectional dimension. A groove or channel 83 extends between the lower and upper end-cap portions at the approximate center of the end-cap near side (e.g., as viewed in FIG. 24). The channel forms a ridge on the corresponding interior surface of the end-cap to secure the end-cap to an end of a radiation source. Substantially cylindrical pins 76, preferably four, are disposed on an exterior near side surface of end-cap 72 and extend transversely away from the end-cap. The pins accommodate wiring from the radiation source and interface a power connector disposed within receptacle 264 of assembly 244 as described above. The transversely extending pins of end-cap 72 enable the radiation sources to be placed within and removed from assembly 244 in a substantially horizontal manner, thereby permitting replacement of the radiation sources without removing assembly 244 from the ceiling or wall unit. Assembly front and rear walls 256, 258 (FIG. 23) typically include slots or grooves to permit placement and removal of radiation sources from assembly 244 in this fashion. The end-cap and pins may be of any size or shape, whereby the end-cap may include any quantity of pins disposed anywhere on the end-cap. Alternatively, radiation sources 36, 62 may be implemented by conventional or other radiation sources utilizing an adapter to interface power connectors within power receptacles 264 in substantially the same manner described above. The adapter may be similar in configuration to the end-cap described above, or include any adapter capable of interfacing a radiation source to the power connector within receptacle 264.

Referring back to FIG. 23, assembly 244 is disposed within the chamber block recess forming the germicidal chambers as described above. Top wall 254 is disposed toward the recess bottom, while rear wall 258 is positioned toward the rear portion of the recess with front wall 256 disposed adjacent the ozone chambers. Ozone sections 12 of combination radiation sources 36 extend through openings 260 in assembly front wall 256 into respective ozone chambers 8a, 8b, via a gap provided in the chamber block between the ozone and germicidal chambers, to provide necessary radiation to generate ozone as described above. A germicidal section 14 of a radiation source 36 and an adjacent radiation source 62 of assembly 244 are disposed within each germicidal chamber. Thus, each germicidal chamber includes a germicidal section of the combination radiation source and an additional radiation source to generate the required germicidal radiation. Since the germicidal chambers share a common area, the radiation sources disposed on assembly 244 combine to remove contaminants and ozone from the air streams received from the respective ozone chambers. Chamber block 242 may be constructed of a light colored or white foam having sufficient reflective properties to reflect radiation from the radiation sources within the ozone and germicidal chambers. The reflective property of the ozone and germicidal chambers increases radiation intensity to enhance the effects of the ozone generating and germicidal radiation described above.

Chamber block 242, having assembly 244 disposed therein as described above, is placed on the base platform wherein cover 240 is placed over the chamber block and attached to the base. Cover 240 is typically constructed of injection molded plastic or other suitably sturdy material, and includes substantially rectangular top, front, rear and side walls 284, 285, 286, 287, respectively, that collectively define the cover interior. The bottom portions of the front, rear and side walls include a ledge 88 transversely extending from the respective walls to enable attachment of the cover to the base. The cover interior includes dimensions slightly larger than chamber block 242 to receive and cover the chamber block as described above. System 2f is typically installed within a ceiling or wall, whereby air enters the system via intake 48 and sterilized air is returned to the environment via exhaust vent 50 (e.g., as indicated by the arrows in FIG. 23) as described above. The air flow path through system 2f is substantially similar to the air flow paths through the corresponding systems described in the aforementioned patent applications. By way of example only, the system typically includes a length of approximately twenty-four inches, a width of approximately twenty-four inches, and an approximate height of eight inches.

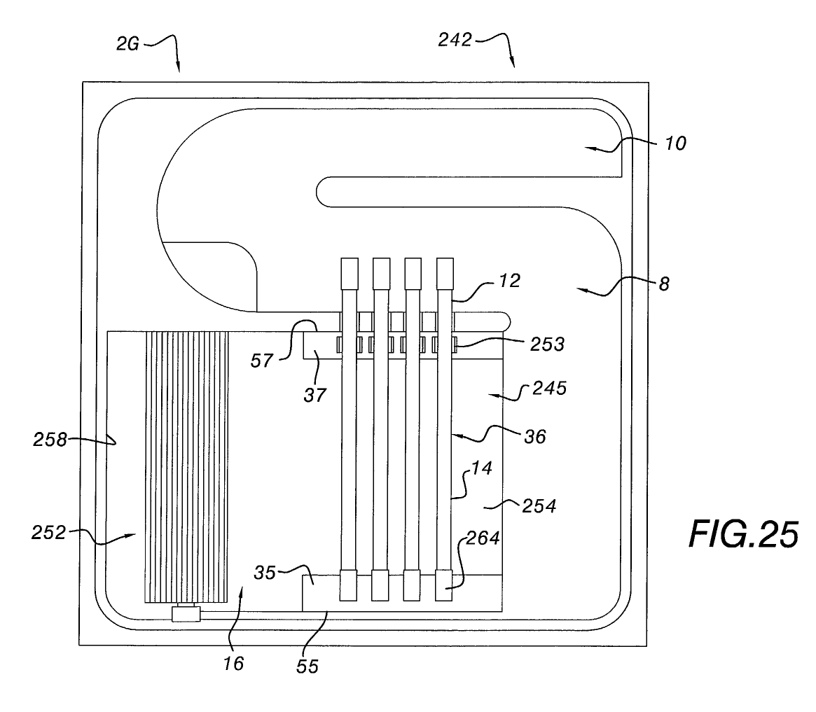

An alternative configuration for system 2f, including a single ozone chamber and a single germicidal chamber, is illustrated in FIG. 25. Specifically, system 2g is substantially similar to and functions in substantially the same manner as system 2f described above for FIG. 23 except that system 2g includes a single ozone chamber and a single germicidal chamber, whereby the germicidal chamber includes a modified assembly 245 having four radiation sources 36. However, any quantity (e.g., at least one) of radiation sources may be utilized. It is to be understood that the system illustrated in FIG. 25 is inverted relative to the system shown in FIG. 23, however, the system of FIG. 25 is typically mounted in substantially the same manner and at substantially the same orientation as the system described above and shown in FIG. 23. System 2g includes ozone chamber 8 and germicidal chamber 16 having modified assembly 245 disposed therein to provide radiation from radiation sources 36. Ozone chamber 8 includes path 10 defined by a winding groove or channel formed in chamber block 242 to reduce air through-flow velocity and mix generated ozone with the air stream to remove contaminants from the air stream as described above.

Germicidal chamber 16 is defined by a substantially rectangular recess formed in chamber block 242 adjacent ozone chamber 8 as described above, while assembly 245 is substantially similar to assembly 244 described above except that assembly 245 includes a modified configuration to accommodate combination radiation sources 36 and the alternative arrangement of system 2g. In particular, assembly 245 includes top wall 254, rear wall 258, and source walls 55, 57. Top wall 254 has dimensions slightly less than the dimensions of the recess within chamber block 242 forming the germicidal chamber such that assembly 245 is inserted within that recess as described below. Rear wall 258 is substantially rectangular and extends from a top wall rear edge substantially perpendicular to the top wall, while each source wall is substantially rectangular and extends from a side edge of a front portion of the top wall substantially perpendicular to the top wall. Rear wall 258 extends from top wall 254 for a distance substantially similar to the depth of the block recess, while source walls 55, 57 and extend from top wall 254 for approximately one-half the height of the rear wall. The upper portions of each source wall 55, 57 transversely extend toward each other to form respective ledges or shelves 35, 37 in facing relation. Ledge 35 typically includes receptacles 264 that include power connectors for connecting radiation sources 36 to a ballast (not shown) as described above. Ledge 37 includes holders 253 that correspond to and coincide with receptacles 264 on ledge 35. Holders 253 include a resilient substantially semi-circular member and have dimensions slightly less than the cross-sectional dimensions of radiation sources 36. Holders 253 receive portions of radiation sources 36 toward ozone section 12 and resiliently engage the radiation source via the resilient member to provide a snug fit, while receptacles 264 receive the ends of radiation sources 36 adjacent germicidal sections 14 as described above. Fan 252 is disposed on rear wall 258 such that the fan is substantially flush with a recess peripheral edge when assembly 245 is disposed within the recess to draw air through the system as described above.

Assembly 245 is disposed within germicidal chamber 16 with top wall 254 positioned toward the recess bottom, rear wall 258 positioned toward a recess far side edge (e.g., as viewed in FIG. 25), source wall 55 positioned toward the bottom portion of the germicidal chamber and source wall 57 positioned adjacent the ozone chamber. In essence, assembly 245 is disposed in the chamber block recess at an orientation rotated approximately ninety degrees from the orientation of assembly 244 within the chamber block recess described above. Radiation sources 36 are disposed within receptacles 264 and holders 253 as described above and extend beyond the holders into ozone chamber 8 via gaps provided in chamber block 242 between the ozone and germicidal chambers. Radiation sources 36 are disposed such that ozone section 12 extends into ozone chamber 8, while germicidal sections 14 reside within the germicidal chamber to provide the necessary radiation within the respective chambers to remove contaminants from the air stream as described above.

Air flows through the system in substantially the same manner described above for FIG. 23. Initially, air enters ozone chamber 8 and path 10 via an intake (not shown) as described above. Ozone sections 12 of radiation sources 36 emit radiation within the ozone chamber to generate ozone that interacts with the air stream to remove contaminants as described above. Path 10 directs the air stream in a winding fashion through the ozone chamber to enable the generated ozone to mix and interact with the air stream to remove contaminants as described above. Upon traversing path 10, the air stream enters germicidal chamber 16, whereby the germicidal chamber exposes the air stream to germicidal radiation from germicidal sections 14 of radiation sources 36 to remove contaminants and ozone from the air stream as described above. Fan 252 draws air through the system and directs purified air back to the surrounding environment via an exhaust vent (not shown) as described above. Assembly 245 may include any quantity of radiation sources of the combination or single radiation emitting type, and may further accommodate the end-cap and adapter arrangements described above.

In addition, the ceiling or wall unit may be implemented by or as a replaceable cartridge system in substantially the same manner described above for system 2e. Specifically, a base housing system electrical components may be disposed within a ceiling or wall, while a cartridge having the ozone and germicidal chambers and corresponding radiation sources may be connected to the base as described above. The ozone and germicidal chambers may have any of the configurations described above. The cartridge and/or base may be visible, or the cartridge and/or base may be partially or totally hidden and include mechanisms (e.g., guides, tubes, etc.) to draw air into the system and return treated air to a surrounding environment. Alternatively, the cartridge may be utilized in the ceiling or wall without the base and be connected to a power source as described above.

The systems described above may be constructed of any suitable materials, however, certain materials, such as plastics, may be vulnerable to ozone and germicidal radiation. In order to prevent damage to those systems utilizing vulnerable materials, the ozone and germicidal chamber structures may be lined with metallic sheets, a metallic coating or include an additive that enables the structures (e.g., bulb terminals, end-caps, adapters, sleeves, casing, wiring sleeves, chambers, etc.) to withstand ozone and ozone generating and germicidal radiation. Further, the metallic sheets, metallic coating or additive may reflect the ultraviolet energy radiation to increase radiation intensity within the chambers to enhance ozone formation and removal of contaminants.

Microwave energy may be utilized by the systems described above in conjunction with ozone and germicidal radiation to further remove contaminants. Specifically, the systems described above may include a magnetron or other conventional microwave energy generating device disposed within the ozone and/or germicidal chambers, or in an additional microwave chamber disposed anywhere in the system exposing the air stream to microwave energy. Alternatively, the magnetron may be disposed anywhere in the system or external of the system or chambers, whereby generated microwave energy may be directed into the ozone, germicidal and/or microwave chambers. The microwave energy kills bacteria residing in the air stream, while the ozone and germicidal radiation remove contaminants as described above. In addition, radiation source 36 may be implemented by an electrodeless bulb that emits radiation in response to microwave energy. The microwave energy may be generated within or directed into the ozone and/or germicidal chambers to remove contaminants and activate radiation source 36. Alternatively, radiation source 36 may be implemented by independent electrodeless radiation emitting bulbs wherein microwave energy is generated within or directed into both the ozone and germicidal chambers to remove contaminants and activate the respective bulbs. For an example of the structure and operation of electrodeless lamps, reference is made to U.S. Pat. Nos. 3,872,349 (Spero et al), 4,042,850 (Ury et al) and 5,614,151 (LeVay et al), the disclosures of which are incorporated herein by reference in their entireties.

Enhanced contaminant removal from an air stream may be accomplished by disposing filters (e.g., washable or disposable filters) or other devices within the systems described above to remove particles, such as allergens, smoke, or other particles, residing within the air stream. Specifically, the systems may include various conventional or other types of filters disposed at any location within the system. The filters remove smoke and other particles from the air stream, while the system removes other contaminants within the air stream via ozone and germicidal radiation as described above. Preferably, the filters remove particles from the air stream subsequent to sterilization of the air to enable washing or disposal of the filter without an adverse effect on the environment (e.g., only sterilized particles are returned to the environment when a filter is washed or replaced). For an example of utilizing filters to remove particles from air, reference is made to U.S. Pat. Nos. 5,186,903 (Cornwell) and 5,221,520 (Cornwell), the disclosures of which are hereby incorporated by reference in their entireties.

Alternatively, the systems described above may use electrical techniques to remove particles from an air stream. For example, the systems may include a precipitator having plates separated by a particular distance. An air stream passes between the plates, whereby an electrostatic field residing between the plates causes smoke or other particles to separate from the air stream and cling to the plates. The precipitator or plates may be disposed anywhere in the system to remove the particles from the air stream, while the system removes other contaminants within the air stream via ozone and germicidal radiation as described above. Preferably, the precipitator removes particles from the air stream subsequent to sterilization of the air to prevent adverse effects on the environment (e.g., non-sterile particles being returned to the environment) as described above. A particle collection receptacle may be disposed proximate the plates, whereby the plates may be manipulated or vibrated by various techniques, such as ultrasound or mechanical and/or electrical manipulation, to facilitate dislodgment of particles from the plates and into the collection receptacle. The collection receptacle may be filled with water or other liquid to maintain the particles within the receptacle, whereby the collection receptacle is periodically emptied to remove the captured particle contents. The particle removal is particularly suited to a commercial environment, such as stores, restaurants and bars, to purify and remove cigarette and cigar smoke or other particles from the air. However, the particle removal may be suited for any other environment such as homes, medical facilities, etc. It is to be understood that any other conventional techniques for particle removal may be utilized by the systems, such as filtering, charging particles for attraction to a particular structure, or washing the air stream. For an example of electrically removing particles from an air stream, reference is made to U.S. Pat. Nos. 3,785,124 (Gaylord) and 3,788,041 (Gaylord), the disclosures of which are incorporated herein by reference in their entireties.

In addition to the foregoing, the systems described above may remove or reduce contaminants within an air stream, such as bacteria, mold spores and viruses, alone or attached to dust, via electrostatic attraction of the contaminants. In particular, activation of the internal fan, especially within systems utilizing sheet metal or other conductors, enables removal or reduction of contaminants in the air stream. The activation of the fan generates an electrostatic charge that attracts and temporarily maintains contaminants within the air stream on the surface of the fan or the system housing and/or structure. Residual ozone, generated by the system during prior operation and residing within the ozone chamber, may interact with these surfaces to remove microbes attracted to the surfaces (e.g., either attracted directly to the surface or attached to particles attracted to the surfaces). The fan and other system surfaces having a charge accumulation essentially attract particles that also develop an electrostatic charge. This effect may be utilized as a separate operating mode of the systems. For an example of an electric field attracting, removing or reducing contaminants within the air, reference is made to the Gaylord patents described above and to U.S. Pat. No. 3,976,448 (Eng et al) the disclosure of which is incorporated herein by reference in its entirety.

Ozone enriched air may be produced and exhausted from the systems described above, whereby the ozone concentration within the ozone enriched air may be controlled in various fashions. For example, the residence time of air within the ozone and germicidal chambers may be adjusted to produce a desired ozone concentration. The residence time may be controlled via configuration of the path, controlling flow within a vortex chamber, adjusting the size of the chambers or any other techniques. Further, the intensity of radiation in each chamber (e.g., the size of the radiation sources), or the portion of the germicidal radiation source in the ozone chamber may be adjusted to control ozone concentration. Intensity of radiation may be controlled by periodically disabling or shielding the ozone or germicidal radiation source via the sleeve or end-cap as described above to respectively control generation or destruction of ozone. Alternatively, the systems described above may include a single chamber exposing air to various combinations of ozone generating and germicidal radiation to produce either purified air or various levels of ozone enriched air.

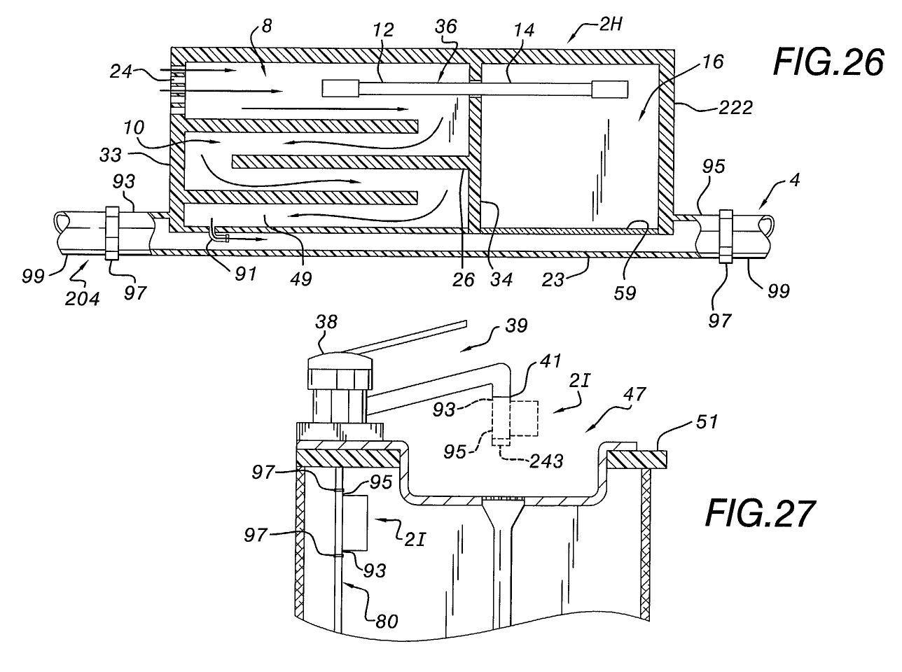

Ozone enriched and/or purified air may be utilized for various applications. For example, since ozone is effective for repelling insects, the systems described above may be configured to produce ozone enriched air and may be placed in rooms, cabinets, closets or other areas. The ozone enriched air produced by the systems may be exhausted from the systems, thereby repelling insects within the surrounding area. Further, the purifying characteristics of ozone enriched air may be utilized to purify liquids, such as tap water flowing to or within houses or buildings, as illustrated in FIG. 26. Specifically, system 2h is similar to and functions in substantially the same manner as the systems described above except that the air and ozone mixture from the ozone chamber is injected into a liquid, while the germicidal chamber exposes the ozone injected liquid to germicidal radiation to remove ozone and contaminants from the liquid in substantially the same manner described above. System 2h includes housing 222, an inlet 93, an outlet 95 and a channel or liquid passage 23 disposed between the inlet and outlet to enable liquid to flow from the inlet through the system to the outlet. Housing 222 is typically substantially rectangular, but may be of any size or shape and may be constructed of any suitable materials (e.g., plastics). The housing includes ozone chamber 8, germicidal chamber 16 and radiation source 36 that each function in substantially the same manner described above. The housing further includes a fan (not shown) and other electrical components (not shown, e.g., ballast, wiring) that draw air through the system and provide power to radiation source 36, respectively. The ballast may be implemented by an A.C. ballast connected to a power line, or a D.C. ballast connected to a battery disposed within the system. Radiation source 36 includes ozone section 12 and germicidal section 14 as described above, and is disposed within housing 222 such that ozone section 12 and germicidal section 14 reside within ozone chamber 8 and germicidal chamber 16, respectively. System 2h is typically disposed along a pipeline 204 directing liquid to various destinations, such as pipes containing tap water extending into houses or other buildings. The system is inserted within pipeline 204 by removing a pipeline section and attaching inlet 93 and outlet 95 to respective pipeline section ends 99 via connectors 97. Connectors 97 may be implemented by any conventional or other connectors forming a liquid tight seal, while the system may alternatively be inserted within the pipeline utilizing any conventional or other techniques, such as welding. Liquid flows through pipeline 204 into channel 23 of system 2h via inlet 93, whereby the system purifies the liquid and directs the liquid back to pipeline 204 via outlet 95 to enable the purified liquid to flow to a pipeline destination as described below. A conventional or other type of filter (not shown) may be disposed toward the inlet or outlet to capture particulate or other matter residing in the liquid.

Ozone chamber 8 is disposed proximate the liquid flow within channel 23 and includes slots 24 defined in housing 222 toward an upper portion of the ozone chamber to receive an air stream from a surrounding environment. The slots may be of any quantity or size and are formed to enable air to enter the system, while maintaining ultra-violet radiation emitted from ozone section 12 within the ozone chamber. An internal fan (not shown) is typically disposed proximate slots 24 and utilized to draw air into the system and through the ozone chamber. Ozone chamber 8 further includes a winding path 10 formed by a series of succeeding passages 49 defined between walls 26 alternately extending from opposing ozone chamber side walls 33, 34. Walls 26 extend across the ozone chamber for a distance slightly less than the distance between side walls 33, 34 to form gaps between walls 26 and side walls 33, 34 to enable the air stream to traverse succeeding passages 49. An air stream enters the ozone via chamber via slots 24 and is exposed to radiation emitted from ozone section 12 to generate ozone within the air stream. The air stream subsequently traverses path 10 wherein the generated ozone mixes and interacts with the air stream to remove contaminants as described above. The path and/or intensity of radiation emitted by ozone section 12 may be adjusted to produce a desired ozone concentration as described above. The ozone enriched air is injected into the liquid flow within channel 23 via a nozzle 91. The nozzle is disposed toward inlet 93 within a passage 49 disposed adjacent channel 23, and injects the ozone enriched air into the liquid to remove contaminants from the liquid in substantially the same manner described above. The liquid flow in combination with nozzle 91 mix the ozone enriched air with the liquid to enable the ozone to remove contaminants as described above. The ozone chamber typically includes a width greater than the width of the germicidal chamber to enable the liquid to flow within channel 23 from the ozone chamber toward the germicidal chamber, thereby facilitating mixing and interaction of the ozone enriched air with the liquid prior to treatment of the liquid by the germicidal chamber. For an example of injecting gases into liquids via nozzles, reference is made to U.S. Pat. Nos. 4,382,866 (Johnson), 4,491,551 (Johnson), 4,562,014 (Johnson), 4,563,286 (Johnson et al) and 4,655,933 (Johnson et al), the disclosures of which are incorporated herein by reference in their entireties.

Subsequent to injection of ozone enriched air into the liquid flowing within channel 23, the liquid flows toward germicidal chamber 16. Germicidal chamber 16 is disposed adjacent ozone chamber 8 and proximate the liquid flowing within channel 23 to expose that liquid to germicidal radiation emitted from germicidal section 14. Germicidal chamber 16 includes a radiation transparent floor 59, preferably constructed of glass or plastic, to maintain liquid within channel 23 (e.g., prevent liquid from entering the germicidal chamber), while enabling germicidal radiation from germicidal section 14 to remove ozone and contaminants from the liquid in substantially the same manner described above.

Further, system 2h may be implemented by or as a replaceable cartridge system in substantially the same manner described above for system 2e. Specifically, a base housing system electrical components and a liquid channel may be disposed along a pipeline as described above, while a cartridge having the ozone and germicidal chambers and corresponding radiation sources may be connected to the base as described above. The ozone and germicidal chambers may contain any of the previously described configurations to expose the liquid to ozone and germicidal radiation as described above.

The system may be disposed along various fluid flows to purify fluid during travel to a particular destination, however, the system is typically utilized to purify tap water flowing into houses, buildings or other structures. In addition, the system may be utilized within these structures to purify tap water flowing to or from a sink faucet as illustrated in FIG. 27. Specifically, system 2i is substantially similar to system 2h described above for FIG. 26, and may be utilized to purify tap water flowing to or from sink faucets, such as sink faucets residing within bathrooms, kitchens, or other locations. A sink 47, typically disposed within a bathroom or kitchen counter 51, includes a faucet 39 having a spout 41 and a handle 38 to control temperature and flow of tap water from the spout into the sink. Faucet 39 typically receives tap water from a pipe 80 disposed within counter 51 that supplies tap water from pipeline 204 (FIG. 26) via a plumbing system (not shown). System 2i may be disposed along pipe 80 in substantially the same manner described above for disposing system 2h along pipeline 204 to purify tap water prior to the tap water flowing through faucet 39.

Alternatively, system 2i may include appropriate dimensions for attachment to faucet 39 proximate spout 41. Specifically, inlet 93 may include dimensions sufficient for connection to spout 41, whereby threads may be disposed on the inlet interior surface for attachment to the spout. However, system 2i may be connected to the spout via any conventional or other fastening techniques or may include larger dimensions and interface spout 41 via an adapter. A conventional aerator 243, typically attached to faucet spouts, may be attached to inlet 93 or outlet 95 to enhance flow into and out of system 2i. Tap water flows from spout 41 into inlet 93 wherein system 2i purifies the tap water in substantially the same manner described above for system 2h as the water flows through the system within channel 23 (FIG. 26) and toward outlet 95.

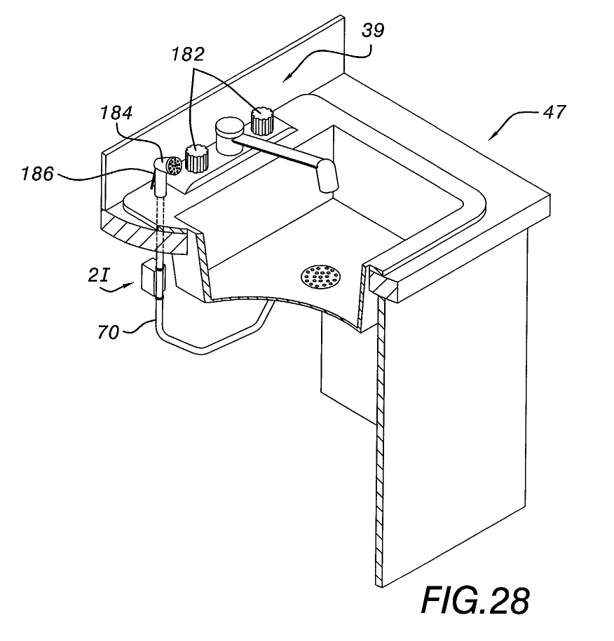

In order to facilitate treatment of food or other items, system 2i may be configured to provide ozonated water to a sink spray nozzle as illustrated in FIG. 28. Specifically, sink 47 is substantially similar to the sink described above except that faucet 39 includes temperature and flow control knobs 182 and sink spray nozzle 184. The nozzle includes a trigger mechanism 186 to enable flow of water from the nozzle. The faucet typically receives tap water from a pipe 80 (FIG. 27) that supplies tap water from pipeline 204 via a plumbing system (not shown). Similarly, nozzle 184 receives water from a pipe 70 connected to either pipe 80 or pipeline 204. System 2i is disposed along pipe 70 in substantially the same manner described above and is typically configured such that the germicidal chamber removes only a portion of ozone from the water, thereby supplying ozonated water to nozzle 184. The ozonated water may be applied, via the nozzle, to various food or other items, such as fruits, vegetables, meat, etc., to remove contaminants from those items. Alternatively, system 2i may be configured to include only the ozone chamber portion to produce ozonated water for nozzle 184.

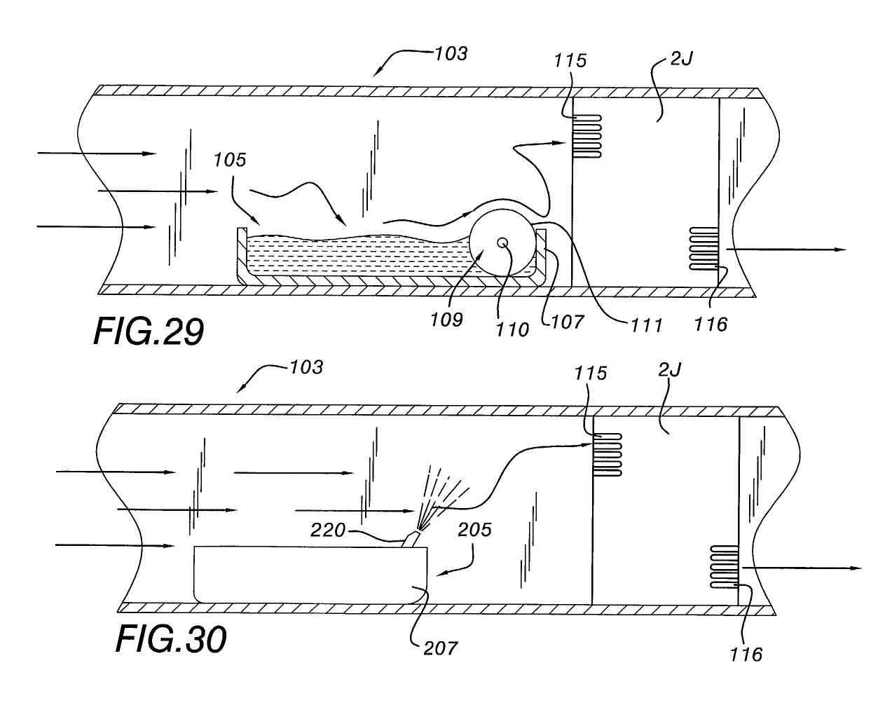

In addition, the systems described above may be employed in various air treatment systems (e.g., HVAC system, humidifier, heating and/or air conditioning units, etc.) to purify air streams within these air treatment systems prior to the air streams returning to a surrounding environment as illustrated in FIG. 29. Specifically, air sterilization system 2j is substantially similar to the air sterilization systems (e.g., systems 2a, 2c-2g ) described above and may be disposed within a duct or compartment 103 of an air treatment system, typically an HVAC system for a house, building, vehicle (e.g., train, airplane, boat, etc.) or other structure. However, system 2j may equally be disposed within humidifiers, heating and/or air conditioning units or any other air treatment systems to remove contaminants from air streams within those systems. Compartment 103 typically includes a humidifier 105 for introducing moisture into an air stream either prior or subsequent to treatment of the air stream by the air treatment system. Humidifier 105 includes a liquid container 107, preferably containing water, and a drum 109 for transferring liquid from liquid container 107 into the air stream. Drum 109 is substantially cylindrical, but may be of any shape, and is disposed within or proximate liquid container 107 in contact with the liquid. A rod or bar 110 is disposed through drum 109 along a drum longitudinal axis to enable the drum to rotate about the rod relative to the liquid residing within liquid container 107. Drum 109 includes a liquid absorbent or sponge type material 111 disposed on and covering the drum exterior surface to absorb the liquid within liquid container 107 as the drum rotates about rod 110. A motor or other mechanical and/or electrical device may be utilized to control rotation of drum 109, whereby the drum typically rotates at a relatively low rate to enable liquid from material 111 to be placed into the air stream. An air stream flowing through compartment 103 interfaces material 111, whereby liquid from liquid container 107 absorbed by the material is introduced into the air stream as the air stream flows by drum 109. Container 107 may be connected to a liquid supply, such as a plumbing system, to enable material 111 to introduce liquid into the air stream.

System 2j includes an air intake vent 115 and an exhaust vent 116 and may be disposed proximate humidifier 105 to remove contaminants from the air stream subsequent to the air stream receiving moisture from drum 109. However, system 2j may be disposed prior to humidifier 105 to remove contaminants from the air stream to enable the humidifier to introduce moisture into a purified air stream. System 2j further includes an ozone chamber as described above disposed toward the upper portion of the system proximate intake vent 115, and a germicidal chamber as described above disposed toward a lower portion of the system proximate exhaust vent 116. The ozone and germicidal chambers and intake and exhaust vents may alternatively be arranged in any fashion. The system receives the air stream from humidifier 105 via intake vent 115 and exposes the air stream to ozone and germicidal radiation in substantially the same manner described above to return purified air to compartment 103 via exhaust vent 116. Humidifier 105 and system 2j may be arranged in any fashion and may be disposed anywhere in the air treatment system either prior or subsequent to treatment of the air stream by the air treatment system (e.g., humidifier 105 and system 2j may be disposed adjacent as described above, or one may be disposed prior to treatment of the air stream by the air treatment system, while the other is disposed subsequent to the air treatment). Further, liquid container 107 may include a germicidal radiation source to expose the liquid within the container to germicidal radiation to remove contaminants from that liquid in substantially the same manner described above for an air stream. In addition, system 2j may be similar in configuration to system 2b (FIG. 9) described above wherein liquid container 107 may utilize germicidal section 14a of radiation source 36 to remove contaminants. For example, liquid container 107 may be disposed within a modified germicidal chamber 16a of system 2b for exposure to germicidal radiation from germicidal section 14a as described above. For examples of utilizing radiation sources to purify liquids, such as water, reference is made to U.S. Pat. Nos. 5,166,527 (Solymar), 5,422,487 (Sauska et al) and 5,614,151 (LeVay et al), the disclosures of which are incorporated herein by reference in their entireties.

Alternatively, compartment 103 may include a humidifier 205 that introduces moisture into the air stream via a spray nozzle as illustrated in FIG. 30. Specifically, compartment 103 and air sterilization system 2j are respectively substantially similar to the compartment and air sterilization system described above for FIG. 29. Compartment 103 typically includes a humidifier 205 for introducing moisture into an air stream and air sterilization system 2j for removing contaminants from the air stream as described above. Humidifier 205 includes a generally enclosed liquid container or tank 207, preferably containing water, and a substantially cylindrical spray nozzle 220 disposed on the liquid container top surface, however, the liquid container may include an open or partially open top portion, while the spray nozzle may be of any shape or size, and may be implemented by any conventional or other type of nozzle. Spray nozzle 220 utilizes liquid residing within liquid container 207 to generate a spray or mist to introduce moisture into the air stream. An air stream flowing through compartment 103 interfaces the mist generated by spray nozzle 220, thereby introducing moisture into the air stream. Liquid container 207 may include a pressure device or pumping mechanism to transfer liquid to the nozzle, and is typically connected to a liquid supply, such as a plumbing system, to maintain generation of the mist.

System 2j is disposed proximate humidifier 205 and receives the air stream from the humidifier via intake vent 115 as described above. The system exposes the air stream to ozone and germicidal radiation in substantially the same manner described above to return purified air to compartment 103 via exhaust vent 116 as described above. Humidifier 205 and system 2j may be arranged in any fashion and may be disposed anywhere in the air treatment system either prior or subsequent to treatment of the air stream by the air treatment system (e.g., humidifier 205 and system 2j may be disposed adjacent as described above, or one may be disposed prior to treatment of the air stream by the air treatment system, while the other is disposed subsequent to the air treatment). Further, liquid container 207 may include a germicidal radiation source to expose the liquid to germicidal radiation to remove contaminants from the liquid as described above. Alternatively, system 2j may be similar in configuration to system 2b (FIG. 9) described above, whereby liquid container 207 may utilize germicidal section 14a of radiation source 36 to remove contaminants in substantially the same manner described above.

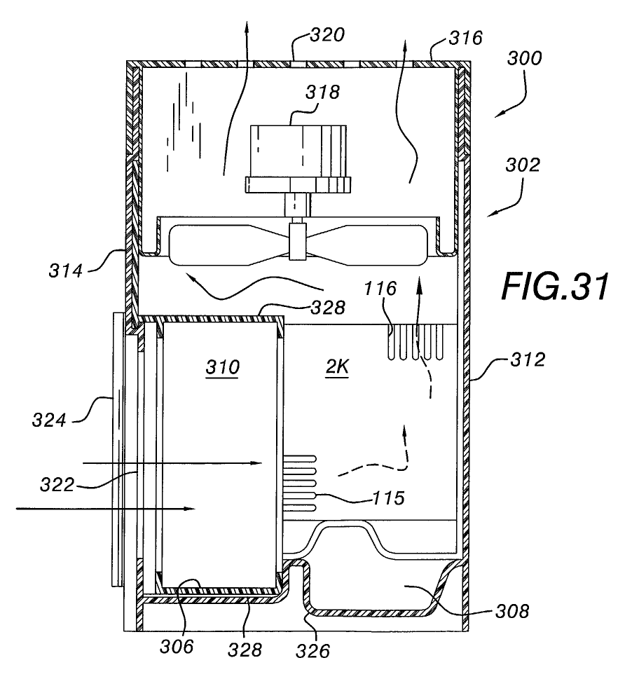

Air sterilization may similarly be utilized within stand alone humidifiers to remove contaminants from an air stream and return purified treated air to surrounding environments, such as rooms or other areas. An exemplary stand alone humidifier including an air sterilization system is illustrated in FIG. 31. Specifically, a stand alone humidifier 300 includes a housing 302, constructed of any suitable materials (e.g., plastics), having a liquid container or receptacle 306, a liquid overflow container 308, a moisture assembly.310 for introducing moisture into an air stream, and an air sterilization system 2k for removing contaminants from the air stream as described below. Housing 302 is substantially rectangular, but may be of any shape, and includes substantially rectangular front, rear and bottom walls 312, 314, 326, respectively, and substantially rectangular side walls (not shown) that collectively define the housing interior. A substantially rectangular cover 316 is disposed on the housing top surface and extends into the housing interior to support a fan 318 that draws air through the humidifier. Cover 316 may alternatively be of any shape and includes slots 320 to permit the air stream to return to a surrounding environment. Slots 320 may be of any size, shape or quantity, and may be arranged in any fashion.

Air enters humidifier 300 via slots 322 defined in a lower portion of housing rear wall 314. Slots 322 may be of any size, shape or quantity, and may be arranged in any fashion. A filter 324, typically conventional, may be disposed on the housing exterior or interior surface coincident slots 322 to initially remove particles and/or contaminants from an incoming air stream. Moisture assembly 310 is disposed coincident, but separated by a slight distance from, slots 322 to introduce moisture into the air stream. Moisture assembly 310 may include any liquid absorbing or other material, or may be implemented by any assembly, preferably a wicking type assembly as known in the art, that is capable of introducing moisture into the air stream. Liquid container 306, preferably containing water, is disposed below slots 322 toward housing bottom wall 326, and may be integral with the housing rear and bottom walls. Moisture assembly 310 is typically disposed within or proximate liquid container 306 in contact with the liquid to draw the liquid into the assembly for introduction into an air stream as the air stream flows through the assembly as described below. Supports 328 suspend moisture assembly 310 proximate slots 322 and direct the air stream from slots 322 into the assembly. Liquid container 306 typically receives liquid from storage containers (not shown) disposed on the housing side walls, while liquid overflow container 308 is disposed adjacent liquid container 306 to receive excess liquid from the liquid container to prevent the liquid container from overflowing. Alternatively, liquid container 306 may be connected to a plumbing system to receive liquid.

System 2k is substantially similar to the air sterilization systems (e.g., systems 2a, 2c-2g) described above and includes an intake vent 115 and exhaust vent 116 as described above. The system includes an ozone chamber as described above disposed toward a lower portion of the system proximate intake vent 115, and a germicidal chamber as described above disposed toward an upper portion of the system proximate exhaust vent 116. However, the ozone and germicidal chambers and intake and exhaust vents may be arranged in any fashion. System 2k is disposed proximate moisture assembly 310 to receive an air stream from the assembly via intake vent 115. The system exposes the air stream to ozone and germicidal radiation in substantially the same manner described above to return purified air to the humidifier via exhaust vent 116. An air guiding mechanism (e.g., a vane, wall, valve, etc., not shown) may be disposed between the assembly and system 2k to direct the air stream to intake vent 115.

In order to enhance purification of the air stream, a germicidal radiation source, such as an ultra-violet (UV) radiation emitting bulb, may be disposed within liquid container 306 and/or liquid overflow container 308 to remove contaminants from the liquid as described above. Further, system 2k may be similar in configuration to system 2b (FIG. 9) described above wherein the liquid and/or overflow containers may utilize germicidal section 14a of radiation source 36 to remove contaminants as described above.

An air stream from a surrounding environment is drawn through filter 324 and slots 322 into humidifier 300 by fan 318. The air stream traverses moisture assembly 310 wherein liquid, preferably water, from liquid container 306 is introduced into the air stream as the air stream flows through the assembly. Subsequent to traversing moisture assembly 310, system 2k removes contaminants from the air stream as described above and returns the purified air stream to the humidifier. Fan 318 draws the purified air through slots 320 in cover 316 to return purified treated air to the surrounding environment. The humidifier components may be arranged or configured in any fashion capable of introducing moisture into an air. stream. Further, the air sterilization system may be disposed anywhere within any type of stand alone or other humidifiers or other air treatment systems to remove contaminants from an air stream within that system as described above. Moreover, the air sterilization system may be of any size or shape and may be configured in any fashion to accommodate an air treatment system. In addition, the air sterilization system may be configured to produce ozone enriched air having a slight ozone concentration level to permit ozone to remove contaminants residing within ducts, compartments or other areas or surfaces of an air treatment system. For examples of the structure and operation of stand alone humidifiers, reference is made to U.S. Pat. Nos. 5,037,583 (Hand), 5,110,511 (Hand), 5,133,904 (Pepper) and 5,250,232 (Pepper et al), the disclosures of which are incorporated herein by reference in their entireties.

It will be appreciated that the embodiments described above and illustrated in the drawings represent only a few of the many ways of implementing a method and apparatus for producing purified or ozone enriched air to remove contaminants from fluids.

The bulb holder system may be of any shape or size, and may be constructed of any suitable materials. The bulb holder system components may be arranged in any manner within the system housing and the base may be implemented by any stand or base capable of supporting that system and its electrical components. The ballasts for the radiation sources may be implemented by any conventional DC (e.g., for portable systems) or AC ballast or other circuitry to supply current to the radiation sources. The radiation source may be implemented by a single bulb or device capable of emitting radiation at the prescribed wavelengths, or independent sources each emitting radiation at a specified wavelength. The system may include any quantity of radiation sources (e.g., at least one) of any shapes disposed in any manner within the system. The bulb holder may be implemented by any gripping or other device capable of manipulating the bulb. The exhaust vent may be of any shape and may be integral with or independent of the bulb holder (i.e., the bulb holder and vent may be implemented by separate devices). The internal fan may be implemented by any quantity of any conventional or other types of fans or devices for drawing air through the system, such as a fan, blower or device to create a differential pressure in the system to cause air flow through the system. The fan or other devices may be disposed in the system in any manner capable of directing air through the system. Further, the fan or devices may include variable flow rates to cause air to flow through the system at various rates. For example, larger areas may require greater flow rates to enable air within these larger areas to be rapidly and efficiently treated by the system. The system may include any quantity (e.g., at least one) of any shaped ozone and germicidal chambers.

The bulb holder system may be constructed by any quantity of pieces having any portion of the system molded therein, whereby the pieces may collectively be attached in any manner to form the system. The bulb connector may be implemented by any conventional or other type of connector. The path may be any path or other configuration capable of reducing air through-flow velocity and enabling the ozone to mix and interact with the air. The ozone chamber may include a portion of the germicidal section of the radiation source to combine the effects of both types of radiation to enhance removal of contaminants. Further, the systems described above may include a catalytic converter or other filter disposed adjacent the germicidal chamber to remove residual ozone from the air stream.

The various ozone and germicidal chamber configurations described above may be of any size or shape, may be oriented in any fashion, may be implemented by any suitable materials, may utilize any of the radiation sources described above, and may be implemented in any of the systems described above. Further, the combination radiation sources described above may include any proportion of ozone section to germicidal radiation section, whereby the ozone section includes a lesser portion of the source than the germicidal section for the various configurations. The combination and independent radiation sources described above may be configured to emit radiation at any desired wavelengths. Moreover, the combination radiation sources described above typically only operate when each section is operable to prevent ozone generation without germicidal radiation to destroy the ozone.

The bulb end-caps may include any configuration or conventional guiding mechanisms to align the end-cap for power or other connections. The power plugs may be of any shape or size, may be implemented by any conventional or other connector, and may include any quantity (e.g., at least one) of receptacles for connecting corresponding pins to a power source. Similarly, the female plug may be implemented by any conventional or other plug, and may include any quantity (e.g., at least one) of extensions or pegs or other configurations to align the end-cap with the power plug. Further, the end-cap may include any quantity (e.g., at least one) of pins of any shape or size and arranged in any fashion to establish power connections for the radiation source.

The ozone-regulating end-caps may include slots, windows or other openings of any quantity (e.g., at least one), shape or size, arranged in any fashion on the end-cap. Further, the slots, windows or other openings of the end-caps may include a radiation transparent covering. It is to be understood that the ozone-regulating end-cap may include any pattern of openings to control emission of ozone generating radiation and production of ozone. Moreover, the ozone-regulating end-caps may include the mechanisms described above for alignment of the end-cap for connections. The alignment and ozone-regulating end-caps may be utilized with independent, combination or other radiation sources.

The systems described above may include any quantity (e.g., at least one) of ozone and germicidal chambers, whereby each chamber may have any suitable configuration, shape or size to treat a fluid. Further, the systems described above may include a single chamber exposing the fluid to ozone and germicidal radiation. Moreover, the systems described above may utilize any quantity of independent radiation sources of any shape or size within each chamber, or any quantity of combination radiation sources of any shape or size having a plurality of sections with each section disposed in and emitting radiation at an appropriate wavelength for a corresponding chamber. The radiation sources may be disposed within the systems described above in any fashion. The fans of the systems described above may be implemented by any quantity of any conventional or other types of fans or devices for drawing air through the systems, such as a fan, blower or device to create a differential pressure in the system to cause air flow through the system. The fans or other devices may be disposed in or external of the systems described above in any manner capable of directing air through the systems. The fan or devices may include variable flow rates to cause air to flow through the systems at various rates. The air flow paths within the ozone and/or germicidal chambers of the systems described above may be any path or other configuration capable of reducing air through-flow velocity and enabling the ozone to mix and interact with the air. The systems described above may include or be connected to any type of ballast or power source, and include any conventional or other corresponding connectors or circuitry. The components of the systems described above may be arranged in any fashion.

The systems employing baffles may include any quantity (e.g., at least one) of baffles within the ozone and germicidal chambers to direct air flow through the systems, and any quantity of additional baffles to maintain radiation within the systems. The radiation limiting baffles may be disposed within the germicidal chamber or at any other suitable location. The baffles may each include various configurations or openings of any quantity (e.g., at least one), shape or size, and may be constructed of any suitable materials. The systems may similarly be of any shape or size, and constructed of any suitable materials.

The cartridges described above may be of any shape or size, and may include any quantity of ozone and germicidal chambers, radiation sources or other system electrical or other components. The radiation sources may be implemented by combination bulbs or independent radiation sources emitting radiation at particular wavelengths. The cartridge ozone and germicidal chambers may include any configurations that reduce through-flow velocity through the system. The posts may be of any quantity, shape or size, and may be disposed in any fashion in the chambers. The chambers may alternatively include any type of obstacle or mechanism to reduce through-flow velocity. The cartridges are preferably disposable and periodically replaced, however, a base and cartridge may be implemented as an integral disposable unit. The base may be of any shape or size, include any quantity (e.g., at least one) of ballasts, fans or other electrical or system components arranged in any fashion, and may be constructed of any suitable materials. The cartridges may each be constructed as a single unit or be formed from any quantity of the same or different components. A base and cartridge may be disposed at any suitable location to treat fluids. The cartridges are preferably constructed of foam, but may be constructed of any suitable materials.

The cartridges may further be utilized without the base and include a connector for receiving power, whereby a cartridge is disposed within a fluid flow that flows through the cartridge, such as in a plenum or duct. The cartridge radiation source end-cap may be of any shape or size capable of displacing the bulb a sufficient distance from the cartridge wall, and may be utilized in any of the cartridge or other system embodiments described above. The cartridge end-cap windows or openings may include a radiation transparent covering. A cartridge with connector may be disposed at any suitable location, such as within walls, ceilings, vehicle plenums, ducts or other locations.

The ceiling or wall unit may be of any size or shape, or constructed of any suitable material and may include any of the ozone and germicidal chamber configurations described above. The ceiling unit may include any quantity of combination and/or independent radiation sources disposed in any manner within the chambers. The electrical assembly may be constructed of any suitable material and may support any quantity of electrical components, fans, radiation sources or other components. Further, the electrical and other components may be disposed on the assembly in any fashion. The fan may be implemented by any quantity of any conventional fans or other types of devices described above and disposed anywhere in the system for directing air through the system. The fans or devices may include variable flow rates as described above. The base may be configured to direct air to and from the chambers in any fashion. The ceiling unit components (e.g., block, cover, base, etc.) may be connected or fastened by any conventional or other fastening techniques. The ceiling unit radiation source end-cap may be of any shape or size and include any quantity of pins of any shape or size disposed at any desired location or orientation.

The systems for removing contaminants from liquids may include any quantity (e.g., at least one) of ozone and germicidal chambers and any quantity (e.g., at least one) of combination or independent radiation sources of any shape or size arranged in any fashion. The ozone chamber may include any suitable configuration to mix ozone with the air stream, while the germicidal chamber may include any configuration to expose the liquid to germicidal radiation. Further, the germicidal radiation source may be disposed at any location, such as within the liquid channel, to expose the liquid to germicidal radiation. The ozone and germicidal chambers may be configured and disposed within the systems in any suitable fashion. The systems may be of any size or shape to accommodate various sized fluid transports, and may be connected to the transports via any conventional or other fastening techniques. The ozone injecting nozzle may be implemented by any conventional or other device for injecting ozone into liquid. The filter may be disposed at any location within the systems to remove particles or other matter from the liquid. The filter may be of any quantity, shape or size, and may be implemented by any conventional or other type of filter for removing particles. The systems may be disposed at any suitable location along a fluid transport. The systems may be configured to produce ozonated liquid for application to various items. The ozone concentration may be controlled by regulating either or both of the ozone generating and germicidal radiation. Alternatively, the systems may include only the ozone chamber to produce ozonated liquid, or the ozone and germicidal chambers may be reversed such that liquid is exposed to germicidal radiation prior to introduction of ozone into the liquid. The systems may be utilized with any type of applicator at any location to ozonate water or other liquid from a liquid supply for application of the ozonated liquid to various objects.

The systems described above may be disposed in air treatment systems, such as HVAC systems, humidifiers, air conditioning and/or heating systems, or other devices to purify air streams within those devices and return purified air to the surrounding environment. The systems may be disposed at any locations within the devices prior, subsequent or during treatment of the air by those devices for purifying an air stream.

It is to be understood that the present invention is not limited to the specific embodiments discussed herein, but may be implemented in any manner that utilizes ozone generation via a configuration that reduces air through-flow velocity to enable the ozone to interact with the air (e.g., any path configuration or other mechanism to reduce air through-flow velocity), and germicidal radiation to remove contaminants from a fluid stream.

From the foregoing description it will be appreciated that the invention makes available a novel method and apparatus for producing purified or ozone enriched air to remove contaminants from fluids wherein air is exposed to UV radiation at a first wavelength to generate ozone while traversing an ozone chamber configured to reduce air through-flow velocity and to enhance ozone distribution in the air. The ozone oxidizes contaminants in a fluid stream, whereby the fluid stream is exposed to UV radiation at a second wavelength to destroy bacteria and ozone in the fluid.

Having described preferred embodiments of a new and improved method and apparatus for producing purified or ozone enriched air to remove contaminants from fluids, it is believed that other modifications, variations and changes will be suggested to those skilled in the art in view of the teachings set forth herein. It is therefore to be understood that all such variations, modifications and changes are believed to fall within the scope of the present invention as defined by the appended claims.

What is claimed is:

1. A system for removing contaminants from a contaminated air stream received from a surrounding environment to produce purified or ozone enriched air comprising:
an air intake to receive an air stream from the surrounding environment;
an ozone chamber including an ozone generating radiation source for irradiating the air stream to produce ozone to remove contaminants from within the air stream, and ozone distribution means for delaying said air stream by increasing residence time of said air stream in said ozone chamber to facilitate interaction and mixing of the produced ozone with the air stream to enhance removal of contaminants from within the air stream;
a germicidal chamber for receiving said air stream from said ozone chamber and including a germicidal radiation source for irradiating the air stream to remove residual contaminants and ozone therefrom;
an exhaust to return the air stream from said germicidal chamber to the surrounding environment; and
air flow control means for directing the air stream to flow through said system.

2. The system of claim 1 further including:
a power source to provide power to said ozone generating radiation source and said germicidal radiation source;
an end-cap disposed at an end of the ozone generating radiation source, wherein said end-cap includes connectors to provide power to said ozone generating radiation source from said power source; and
a guide mechanism to align said end-cap in a proper position to facilitate a connection between said end-cap and said power source.

3. The system of claim 1 further including:
an end-cap disposed coincident at least a portion of the ozone generating radiation source, wherein said end-cap includes a configuration to regulate emission of radiation from said ozone generating radiation source to control production of ozone within the ozone chamber.

4. The system of claim 1 further including:
a germicidal treatment chamber for receiving said air stream from said air intake and including a germicidal treatment radiation source for irradiating the air stream to remove contaminants from within the air stream, wherein said ozone chamber receives said air stream from said germicidal treatment chamber.

5. The system of claim 1 wherein:
said ozone distribution means includes a plurality of ozone chamber baffles collectively configured to direct said air stream in a serpentine fashion through said ozone chamber; and
said germicidal chamber further includes a plurality of germicidal chamber baffles collectively configured to direct said air stream in a serpentine fashion through said germicidal chamber.

6. The system of claim 5 further including:
a plurality of radiation baffles disposed proximate said germicidal chamber and configured to collectively maintain radiation emitted by said germicidal radiation source within said system.

7. The system of claim 1 wherein said surrounding environment is a duct of an air treatment system, and said air intake includes means for receiving said air stream from said air treatment system duct.

8. The system of claim 7 wherein said air treatment system duct includes a humidifier, and said air intake includes means for receiving said air stream from said humidifier.

9. The system of claim 1 wherein said surrounding environment is an interior of a humidifier unit, and said air intake includes means for receiving said air stream from said humidifier unit interior.

10. A system for removing contaminants from a contaminated air stream received from a surrounding environment to produce purified or ozone enriched air comprising:
a replaceable cartridge disposed within said surrounding environment, wherein said cartridge includes:
an air intake to receive an air stream from the surrounding environment;
an ozone chamber including an ozone generating radiation source for irradiating the air stream to produce ozone to remove contaminants from within the air stream, and ozone distribution means for delaying said air stream by increasing residence time of said air stream in said ozone chamber to facilitate interaction and mixing of the produced ozone with the air stream to enhance removal of contaminants from within the air stream;
a germicidal chamber for receiving said air stream from said ozone chamber and including a germicidal radiation source for irradiating the air stream to remove residual contaminants and ozone therefrom;
an exhaust to return the air stream from said germicidal chamber to the surrounding environment; and
a connector to connect said cartridge to a power source.

11. The system of claim 10 further including:
a base for disposal in said surrounding environment, wherein said base includes:
air flow control means for directing the air stream to flow through said system; and
said power source for interfacing said connector to provide power to said system;
wherein said replaceable cartridge is removably attached to said base.

12. The system of claim 10 further including an end-cap disposed at an end of said ozone generating radiation source, wherein said end-cap is configured to maintain said ozone generating radiation source away from walls of said cartridge.

13. A system for producing ozone enriched air to remove contaminants from liquids comprising:
a liquid inlet to receive liquid from a surrounding environment;
a liquid channel for receiving said liquid from said inlet and directing said liquid through said system;
an ozone chamber including:
air flow control means for directing an air stream through said ozone chamber;
an ozone generating radiation source for irradiating the air stream to produce ozone to remove contaminants from within the air stream;
ozone distribution means for delaying said air stream by increasing residence time of said air stream in said ozone chamber to facilitate interaction and mixing of the produced ozone with the air stream to enhance removal of contaminants from within the air stream; and
an injector disposed proximate said liquid channel to introduce ozone from said ozone chamber into said liquid;
a germicidal chamber including a germicidal radiation source for irradiating said ozonated liquid within said liquid channel to remove residual contaminants and at least a portion of ozone therefrom; and
a liquid outlet to return said treated liquid to said surrounding environment.

14. The system of claim 13 further including:
an applicator for receiving said treated liquid from said outlet and applying said treated liquid to objects, wherein said treated liquid includes a predetermined concentration level of ozone and said applicator applies said ozonated liquid to said objects to remove contaminants therefrom.

15. In an air sterilization system having an air intake, ozone and germicidal chambers and an exhaust, a method of removing contaminants from a contaminated air stream received from a surrounding environment to produce purified or ozone enriched air comprising the steps of:
(a) receiving an air stream from the surrounding environment;
(b) directing the air stream to flow through the system;
(c) irradiating the air stream within the ozone chamber via an ozone generating radiation source to produce ozone to remove contaminants from within the air stream;
(d) delaying said air stream by increasing residence time of said air stream in said ozone chamber to facilitate interaction and mixing of the produced ozone with the air stream to enhance removal of contaminants from within the air stream;
(e) irradiating the air stream received from the ozone chamber within a germicidal chamber via a germicidal radiation source to remove residual contaminants and ozone therefrom; and
(f) returning the air stream from said germicidal chamber to the surrounding environment.

16. The method of claim 15 wherein said air sterilization system further includes a power source to provide power to said ozone generating radiation source and said germicidal radiation source, wherein said ozone generating radiation source includes an end-cap having connectors to provide power to said ozone generating radiation source from said power source, and step (c) further includes:
(c.1) aligning said end-cap in a proper position, via a guiding mechanism, to facilitate a connection between said end-cap and said power source.

17. The method of claim 15 wherein an end-cap is disposed coincident at least a portion of the ozone generating radiation source, and step (c) further includes:
(c.1) regulating emission of radiation from said ozone generating radiation source via said end-cap to control production of ozone within the ozone chamber.

18. The method of claim 15 wherein said air sterilization system further includes a germicidal treatment chamber, and step (b) further includes:
(b.1) receiving said air stream from said surrounding environment and irradiating the air stream within the germicidal treatment chamber via a germicidal treatment radiation source to remove contaminants from within the air stream;
and step (c) further includes:
(c.1) receiving said air stream from said germicidal treatment chamber.

19. The method of claim 15 wherein said ozone chamber includes a plurality of ozone chamber baffles, said germicidal chamber includes a plurality of germicidal chamber baffles, and step (c) further includes:
(c.1) directing said air stream in a serpentine fashion through said ozone chamber via said ozone chamber baffles; and
step (e) further includes:
(e.1) directing said air stream in a serpentine fashion through said germicidal chamber via said germicidal chamber baffles.

20. The method of claim 19 wherein said air sterilization system further includes a plurality of radiation baffles disposed proximate said germicidal chamber, and step (e) further includes:
(e.2) maintaining radiation emitted by said germicidal radiation source within said system via said radiation baffles.

21. The method of claim 15 wherein said surrounding environment is a duct of an air treatment system, and step (a) further includes:
(a.1) receiving said air stream from said air treatment system duct.

22. The method of claim 21 wherein said air thermal treatment system duct includes a humidifier, and step (a.1) further includes:
(a.1.1) receiving said air stream from said humidifier.

23. The method of claim 15 wherein said surrounding environment is an interior of a humidifier unit, and step (a) further includes:
(a.1) receiving said air stream from said humidifier unit interior.

24. In an air sterilization system including a replaceable cartridge having an air intake, ozone and germicidal chambers, an exhaust and a connector to connect the cartridge to a power source, a method of removing contaminants from a contaminated air stream received from a surrounding environment to produce purified or ozone enriched air comprising the steps of:
(a) disposing the cartridge within said surrounding environment;
(b) interfacing the connector to the power source;
(c) receiving an air stream into the cartridge from the surrounding environment;
(d) irradiating the air stream within the ozone chamber to produce ozone to remove contaminants from within the air stream;
(e) delaying said air stream by increasing residence time of said air stream in said ozone chamber to facilitate interaction and mixing of the produced ozone with the air stream to enhance removal of contaminants from within the air stream;
(f) irradiating the air stream received from the ozone chamber within the germicidal chamber to remove residual contaminants and ozone therefrom;
(g) returning the air stream from said germicidal chamber to the surrounding environment; and
(h) periodically replacing the cartridge within the surrounding environment.

25. The method of claim 24 wherein said air sterilization system further includes a base having air flow control means for directing air through said system and said power source, and step (a) further includes:
(a.1) disposing the base within the surrounding environment;
(a.2) removably attaching the cartridge to the base; and
(a.3) directing the air stream through the system via the air flow control means.

26. The method of claim 24 wherein the air sterilization system further includes an end-cap disposed at an end of said ozone generating radiation source, and step (d) further includes:
(d.1) maintaining said ozone generating radiation source away from walls of said cartridge via said end-cap.

27. In a liquid sterilization system having an inlet, a liquid channel, ozone and germicidal chambers, an injector and an outlet, a method of producing ozone enriched air to remove contaminants from liquids comprising the steps of:
(a) receiving liquid from a surrounding environment;
(b) directing said liquid from said inlet through said system via said liquid channel;
(c) directing air from the surrounding environment through said ozone chamber;
(d) irradiating the air stream within the ozone chamber to produce ozone to remove contaminants from within the air stream;
(e) delaying said air stream by increasing residence time of said air stream in said ozone chamber to facilitate interaction and mixing of the produced ozone with the air stream to enhance removal of contaminants from within the air stream;
(f) introducing ozone from said ozone chamber into said liquid in said liquid channel via said injector;
(g) irradiating said ozonated liquid within said liquid channel in the germicidal chamber to remove residual contaminants and at least a portion of ozone therefrom; and
(h) returning said treated liquid to said surrounding environment.

28. The method of claim 27 wherein said liquid sterilization system further includes an applicator for receiving said treated liquid from said outlet, wherein said treated liquid includes a predetermined concentration level of ozone, said method further including the step of:
(i) applying said ozonated liquid to objects via said applicator to remove contaminants therefrom.
